(19) United States
(12) Patent Application Publication
(10) Pub. No.: US 2017/0071903 A1
(43) Pub. Date: Mar. 16, 2017

(54) USE OF ERIBULIN AND MTOR INHIBITORS AS COMBINATION THERAPY FOR THE TREATMENT OF CANCER

(71) Applicant: Eisai R&D Management Co., Ltd., Tokyo (JP)

(72) Inventors: Yasuhiro FUNAHASHI, Boston, MA (US); Bruce A. Littlefield, Andover, MA (US); Toshimitsu UENAKA, West Chester, PA (US)

(21) Appl. No.: 15/123,476

(22) PCT Filed: Mar. 2, 2015
(86) PCT No.: PCT/US15/18335

§ 371 Date: Sep. 2, 2016

Related U.S. Application Data
(60) Provisional application No. 61/947,398, filed on Mar. 3, 2014.

(51) Int. Cl. A61K 31/357; A61K 45/06; A61K 31/436

(52) U.S. Cl. CPC A61K 31/357; A61K 31/436; A61K 45/06

(57) ABSTRACT

Methods for treating cancer (e.g., breast cancer, lung cancer, pancreatic cancer, primitive neuroectodermal tumors, lung cancer, ovarian cancer, endometrial cancer, pharyngeal cancer, esophageal cancer, and sarcoma) in a subject (such as an human patient) in need thereof by administering eribulin (e.g., eribulin mesylate, i.e., E7389, Halaven) in combination with one or more mammalian target of rapamycin (mTOR) inhibitors (e.g., everolimus, ridaforolimus, and temsirolimus), and kits therefor are provided.

### BACKGROUND OF THE INVENTION

Cancer encompasses a wide variety of diseases that are each characterized by the uncontrolled growth of a particular type of cell. It begins in a tissue containing such a cell and, if the cancer has not spread to any additional tissues at the time of diagnosis, may be treated by, for example, surgery, radiation, or another type of localized therapy. However, when there is evidence that cancer has metastasized from its tissue of origin, different approaches to treatment are typically used. Indeed, because it is not possible to determine with certainty the extent of metastasis, systemic approaches to therapy are usually undertaken when any evidence of spread is detected. These approaches can involve the administration of chemotherapeutic drugs that interfere with the growth of rapidly dividing cells, such as cancer cells. Other approaches involve the use of immunotherapy, in which an immune response against cancerous cells in a subject is elicited or enhanced.

Halichondrin B is a structurally complex, macrocyclic compound that was originally isolated from the marine sponge Halichondria okadai, and subsequently was found in Axinella sp., Phakellia carteri, and Lissodendoryx sp. A total synthesis of halichondrin B was published in 1992 (Aicher et al., J. Am. Chem. Soc. 114:3162-3164, 1992). Halichondrin B has been shown to inhibit tubulin polymerization, microtubule assembly, beta^{S}-tubulin crosslinking, GTP and vinblastine binding to tubulin, and tubulin-dependent GTP hydrolysis in vitro. This molecule has also been shown to have anti-cancer properties in vitro and in vivo. Halichondrin B analogs having anti-cancer activities are described in U.S. Pat. No. 6,214,865 B1.

Eribulin is a synthetic analog of halichondrin B. Eribulin is also known as ER-086526, and has been assigned CAS number 253128-41-5 and US NCI designation number NSC-707389. The mesylate salt of eribulin (eribulin mesylate, which is marketed under the trade name HALAVEN® and is also known as E7389) is approved for the treatment of patients with breast cancer who have previously received at least two chemotherapeutic regimens for the treatment of metastatic disease that should have included an anthracycline and a taxane in either the adjuvant or metastatic setting.

The chemical name for eribulin mesylate is II, 15:18,21:24,28-triepoxy-7,9-ethano-12,15-methano-9H,15H-furo[3,2-i]furo[2′,3′:5,6]pyrano[4,3-b][1,4]dioxacyclopentacosin-5(4H)-one, 2-[(2S)-3-amino-2-hydroxypropyl]hexacosahydro-3-methoxy-26-methyl-20,27-bis(methylene)-, (2R,3R,3aS,7R,8aS,9S,10aR,11S,12R,13aR,13bS,15S,18S,21S,24S,26R,28R,29aS)-methanesulfonate (salt), and it can be depicted as follows:

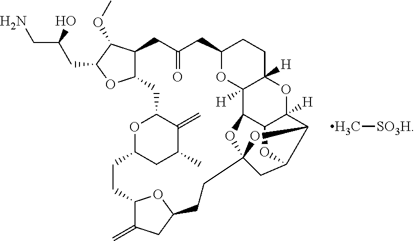

mTOR (also known as mammalian target of rapamycin, mechanistic target of rapamycin, and FK506-binding protein 12-rapamycin-associated protein 1 (FRAP1)) is a serine/threonine kinase that regulates cell growth, cell proliferation, cell motility, cell survival, protein synthesis, and transcription. mTOR belongs to the phosphatidylinositol 3-kinase-related kinase protein family. mTOR Complex 1 (mTORC1) is composed of mTOR, regulatory-associated protein of mTOR (raptor), mammalian lethal with SEC13 protein 8 (MLST8), and the non-core components PRAS40 and Deptor. This complex functions as a nutrient/energy/redox sensor and also plays a role in controlling protein synthesis.

Everolimus (RAD-001) is an inhibitor of mTOR and it exerts its effect on the mTORC1 complex. Everolimus is marketed by Novartis under the tradename Afinitor in oncology. The chemical name for everolimus is dihydroxy-12-[(2R)-1-[(1S,3R,4R)-4-(2-hydroxyethoxy)-3-methoxycyclohexyl]propan-2-yl]-19,30-dimethoxy-15,17,21,23,29,35-hexamethyl-11,36-dioxa-4-azatricyclo[30.3.1.0 hexatriaconta-16,24,26,28-tetraene-2,3,10,14,20-pentone, and it can be depicted as follows:

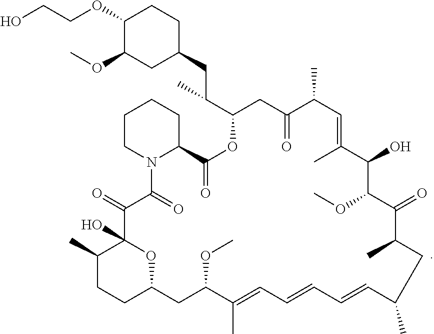 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OCCO)[C@H](OC)C2)CC(=O)[C@H](C)/C=C(\C)[C@@H](O)[C@@H](OC)C(=O)[C@H](C)C[C@H](C)/C=C/C=C/C=C/1C]

### SUMMARY OF THE INVENTION

The invention is based on the observation that the combination of eribulin mesylate and an mTOR inhibitor, everolimus, shows improved (e.g., synergistic) antitumor effects. Therefore, the present invention features methods of preventing and treating cancer by use of the combination of eribulin (e.g., eribulin mesylate) and one or more mTOR inhibitors (e.g., everolimus).

When the term “eribulin” is used herein, it should be considered as indicating eribulin or a pharmaceutically acceptable salt thereof (such as eribulin mesylate), unless the context indicates otherwise. Similarly, when the term “mTOR inhibitor” (or the name of a specific mTOR inhibitor, such as everolimus) is used herein, it should be considered as indicating the mTOR inhibitor or a pharmaceutically acceptable salt, hydrate, solvate, or amorphous solid thereof, as applicable, unless the context indicates otherwise.

The invention provides methods for treating a subject (e.g., a human patient) having or at risk of developing cancer. These methods include administering to the subject (i) eribulin or a pharmaceutically acceptable salt thereof (e.g., eribulin mesylate), and (ii) an inhibitor of mammalian target of rapamycin (mTOR) (e.g., everolimus, ridaforolimus, or temsirolimus) or a pharmaceutically acceptable salt, hydrate, solvate, or amorphous solid thereof. The subject can be diagnosed with a cancer, in treatment for cancer, or in post-therapy recovery from cancer. In various embodiments, the cancer can be a primary tumor or a metastasis, and can optionally be a solid tumor. In further embodiments, the cancer is selected from the group consisting of breast cancer, lung cancer, pancreatic cancer, primitive neuroectodermal tumors, lung cancer, ovarian cancer, endometrial cancer, pharyngeal cancer, esophageal cancer, and sarcoma.

Eribulin or a pharmaceutically acceptable salt thereof (e.g., eribulin mesylate) can be administered by intravenous infusion for, e.g., 1 to about 20 minutes, e.g., for about 2 to about 5 minutes. The amount of eribulin or a pharmaceutically acceptable salt thereof (e.g., eribulin mesylate) can be in the range of about 0.1 mg/m² to about 20 mg/m² (e.g., 1.4 mg/m² or 1.1 mg/m²), optionally administered once daily on each of days 1 and 8 of a 21-day cycle, or once daily on each of days 1 and 15 of a 28-day cycle.

The mTOR inhibitor (e.g., everolimus, ridaforolimus, or temsirolimus) can be administered orally in, e.g., an amount in the range of about 0.1 mg to about 30 mg (e.g., 10 mg), and can optionally be administered once daily during a 21-day cycle or a 28-day cycle.

Eribulin or a pharmaceutically acceptable salt thereof, and an mTOR inhibitor (e.g., everolimus, ridaforolimus, or temsirolimus), or a pharmaceutically acceptable salt, hydrate, solvate, or amorphous solid thereof, can be administered substantially simultaneously or sequentially. For example, eribulin or a pharmaceutically acceptable salt thereof can be administered prior to an mTOR inhibitor (e.g., everolimus, ridaforolimus, or temsirolimus), or a pharmaceutically acceptable salt, hydrate, solvate, or amorphous solid thereof.

Furthermore, eribulin or a pharmaceutically acceptable salt thereof (e.g., eribulin mesylate), and an mTOR inhibitor (e.g., everolimus, ridaforolimus, or temsirolimus), or a pharmaceutically acceptable salt, hydrate, solvate, or amorphous solid thereof, can optionally be administered as sole anti-cancer agents.

Treatment according to the methods of the invention: (i) reduces the number of cancer cells; (ii) reduces tumor volume; (iii) increases tumor regression rate; (iv) reduces or slows cancer cell infiltration into peripheral organs; (v) reduces or slows tumor metastasis; (vi) reduces or inhibits tumor growth; (vii) prevents or delays occurrence and/or recurrence of the cancer and/or extends disease- or tumor-free survival time; (viii) increases overall survival time; (ix) reduces the frequency of treatment; and/or (x) relieves one or more of symptoms associated with the cancer.

The invention also provides methods for decreasing the size of a tumor in a subject (e.g., a human patient). These methods include administering to the subject (i) eribulin or a pharmaceutically acceptable salt thereof (e.g., eribulin mesylate), and (ii) an mTOR inhibitor (e.g., everolimus, ridaforolimus, or temsirolimus) or a pharmaceutically acceptable salt, hydrate, solvate, or amorphous solid thereof. These methods can involve the administration regimens and cancers described above and elsewhere herein.

Also included in the invention are kits for use in preventing or treating cancer or decreasing tumor size (also see, e.g., the effects listed above). The kits can include (i) eribulin or a pharmaceutically acceptable salt thereof, and (ii) an mTOR inhibitor (e.g., everolimus, ridaforolimus, or temsirolimus) or a pharmaceutically acceptable salt, hydrate, solvate, or amorphous solid thereof, optionally in dosage form.

The invention further includes pharmaceutical compositions including the agents noted herein for use in preventing and treating the diseases and conditions noted herein. Furthermore, the invention includes use of the agents noted herein for preparing medicaments and/or for preventing or treating these diseases and conditions.

The methods of the invention provide improved efficacy against cancer. For example, the combination treatment methods described herein can be used to obtain synergistic effects in which, for example, the effects are greater than the sum of the effects of the drugs administered individually, as can be determined by those of skill in the art. Additive effects are also beneficial.

Other features and advantages of the invention will be apparent from the following detailed description, drawings, and claims.

### BRIEF DESCRIPTION OF THE DRAWINGS

FIG. 1 is a graph showing the effect of treatment with E7389 on mean body weight of female athymic nude mice implanted SC with MX-1 mammary tumor xenografts.

FIG. 2 is a graph showing the response of SC implanted MX-1 mammary tumor xenografts to treatment with E7389.

FIG. 3 is a graph showing the effect of treatment with everolimus on mean body weight of female athymic nude mice implanted SC with MX-1 mammary tumor xenografts.

FIG. 4 is a graph showing the response of SC implanted MX-1 mammary tumor xenografts to treatment with everolimus.

FIG. 5 is a graph showing the effect of treatment with E7389 in combination with everolimus (40 mg/kg/dose) on mean body weight of female athymic nude mice implanted SC with MX-1 mammary tumor xenografts.

FIG. 6 is a graph showing the effect of treatment with E7389 in combination with everolimus (20 mg/kg/dose) on mean body weight of female athymic nude mice implanted SC with MX-1 mammary tumor xenografts.

FIG. 7 is a graph showing the response of SC implanted MX-1 mammary tumor xenografts to treatment with E7389 (0.6 mg/kg/inj) in combination with everolimus.

FIG. 8 is a graph showing the response of SC implanted MX-1 mammary tumor xenografts to treatment with E7389 (0.4 mg/kg/inj) in combination with everolimus.

FIG. 9 is a graph showing the response of SC implanted MX-1 mammary tumor xenografts to treatment with E7389 (0.2 mg/kg/inj) in combination with everolimus.

FIG. 10 illustrates quantification of Loewe Volume using Loewe Additivity. Synergy can be quantified in relation to the Loewe additivity shape model which is constructed from the single agent dose responses. The additivity model serves as a “null-hypothesis” and assumes no synergistic interaction between chemical A and B. As noted in the figure, the highest effect level at any concentration dictates the shape of the model. To quantify Loewe Volume, the empiric data surface is subtracted from the additivity shape model. Loewe Volume is the summation of any residual excess activity across the combination dose matrix.

FIG. 11 shows the GI₅₀ of eribulin across a panel of twenty-five cell lines. The median GI₅₀ across the cell line panel is 0.51 nM. Single agent dose analysis was performed in 1536-well and 384-well plate formats using a three-fold, ten-point dose titration. The top histogram displays the cell line data by increasing sensitivity to eribuilin. The bottom histogram displays eribuilin single agent activity across the various tumor types.

FIG. 12 illustrates 6×6 dose matrix format. An enhancer single agent dose curve is shown on the vertical axis. An enhancee single agent dose curve is shown along the horizontal axis. Each enhancee and enhancer is collected in a single agent dose series of five points, while a total of twenty-five combination dose ratio points are collected in the 6×6 dose matrix.

FIG. 13 shows synergy score heat map values for everolimus. The analysis was performed using six breast (MCF7, MDA-MB-231, MDA-MB-436, MDA-MB-468, SK-BR-3, and T47D), six lung (A549, NCI-H1650, NCI-H460, NCI-H522, NCI-H526, and NCI-H69), two ovarian (A2780 and SK-OV-3), and a sampling of single representative cell lines of other tumor types. The twenty-five cell lines are depicted across the horizontal axis. A Synergy Score cut-off of 4.42 was determined for this analysis. Combination activities (growth inhibition data) for the combination of everolimus×eribulin in MDA-MB-468, T47D, NCI-H69, A2780, FaDu, and HT-1080 cells are shown.

FIG. 14 shows synergy score values and Loewe Volume scores for everolimus in the indicated cell types.

### DETAILED DESCRIPTION OF THE INVENTION

The invention provides methods for the prevention and treatment of cancer involving administration of eribulin or a pharmaceutically acceptable salt thereof (e.g., eribulin mesylate) and one or more mTOR inhibitors (e.g., everolimus). Treatment of cancer by administering eribulin or a pharmaceutically acceptable salt thereof (e.g., eribulin mesylate) and everolimus, according to the methods of the invention, can (i) reduce the number of cancer cells; (ii) reduce tumor volume; (iii) increase tumor regression rate; (iv) reduce or slow cancer cell infiltration into peripheral organs; (v) reduce or slow tumor metastasis; (vi) reduce or inhibit tumor growth; (vii) prevent or delay occurrence and/or recurrence of the cancer and/or extend disease- or tumor-free survival time; (viii) increase overall survival time; (ix) reduce the frequency of treatment; and/or (x) relieve one or more of symptoms associated with the cancer.

### Pharmaceutical Compositions, Dosage, and Methods

Methods for the synthesis of eribulin are described, for example, in U.S. Pat. No. 6,214,865; U.S. Pat. No. 7,982,060; U.S. Pat. No. 8,350,067; and U.S. Pat. No. 8,093,410, each of which is incorporated herein by reference. As noted above, eribulin mesylate is available commercially and is marketed as HALAVEN®. Methods relating to everolimus and its synthesis are described, for example, in U.S. Pat. Nos. 5,665,772, 6,004,973, 7,297,703, 8,410,131, 8,436,010, which are incorporated herein by reference. Also, as noted above, everolimus is marketed as AFINITOR®. As discussed further below, mTOR inhibitors in addition to everolimus can also be used in the invention and are available commercially or can be synthesized using methods known in the art.

As noted above, eribulin and/or an mTOR inhibitor (e.g., everolimus) can optionally be used in the present invention in salt forms. There are no particular limitations as to the salt used, whether inorganic acid salt or organic acid salt. For example, the salt can be selected from mesylic acid salt (e.g., eribulin mesylate), hydrochloric acid salt, sulfuric acid salt, citrate, hydrobromic acid salt, hydroiodine acid salt, nitric acid salt, bisulfate, phosphoric acid salt, super phosphoric acid salt, isonicotinic acid salt, acetic acid salt, lactic acid salt, salicic acid salt, tartaric acid salt, pantotenic acid salt, ascorbic acid salt, succinic acid salt, maleic acid salt, fumaric acid salt, gluconic acid salt, saccharinic acid salt, formic acid salt, benzoic acid salt, glutaminic acid salt, methanesulfonic acid salt, ethanesulfonic acid salt, benzenesulfonic acid salt, p-toluenesulfonic acid salt, pamoic acid salt (pamoate), and so on. Moreover, it is acceptable to use salt of aluminum, calcium, lithium, magnesium, sodium, zinc, and diethanolamine.

Pharmaceutical compositions including eribulin and/or an mTOR inhibitor (e.g., everolimus) can be prepared using standard methods known in the art (see, e.g., the patent documents noted above). Typically, eribulin and an mTOR inhibitor (e.g., everolimus) as used in the invention are included within separate pharmaceutical compositions but they can, optionally, be included within a single composition. Eribulin is typically provided in liquid form, for intravenous administration, while an mTOR inhibitor (e.g., everolimus) is typically provided in tablet form, for oral administration.

Pharmaceutical compositions used in the invention can be prepared by, for example, mixing or dissolving the active ingredient(s), having the desired degree of purity, in a physiologically acceptable diluent, carrier, excipient, or stabilizer (see, e.g., Remington's Pharmaceutical Sciences (20^{th} edition), ed. A. Gennaro, 2000, Lippincott, Williams & Wilkins, Philadelphia, Pa.). Acceptable diluents include water and saline, optionally including buffers such as phosphate, citrate, or other organic acids; antioxidants including butylated hydroxytoluene (BHT), butylated hydroxyanisole (BHA), ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, or other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

In preparing compositions for oral dosage form (e.g., compositions including an mTOR inhibitor, such as everolimus), any of the usual pharmaceutical media can be employed, for example, water, glycols, oils, alcohols, flavoring agents, preservatives, coloring agents. In addition, carriers such as starches, sugars, microcristalline cellulose, diluents, granulating agents, lubricants, binders, disintegrating agents, and the like can be used in the case of oral solid preparations such as, for example, powders, capsules, and tablets.

Optionally, the formulations of the invention contain a pharmaceutically acceptable preservative. In some embodiments the preservative concentration ranges from 0.1 to 2.0%, typically v/v. Suitable preservatives include those known in the pharmaceutical arts, such as benzyl alcohol, phenol, m-cresol, methylparaben, and propylparaben. Further, the eribulin and/or mTOR inhibitor (e.g., everolimus) formulations can optionally include a pharmaceutically acceptable salt, such as sodium chloride at, for example, about physiological concentrations. Thus, in one example, eribulin (e.g., eribulin mesylate) is formulated in 0.9% Sodium Chloride Injection (USP).

The formulations noted above (and others) can be used for parenteral administration of the drugs. Thus, the drugs can be administered by routes including intravenous, intra-tumoral, peri-tumoral, intra-arterial, intra-dermal, intra-vesical, ophthalmic, intramuscular, intradermal, intraperitoneal, pulmonary, subcutaneous, and transcutaneous routes. Other routes can also be used including, for example, transmucosal, transdermal, inhalation, intravaginal, rectal, and oral administration routes.

The dosage of the eribulin and/or mTOR inhibitor (e.g., everolimus) compositions administered can differ markedly depending on the type of target disease, the choice of delivery method, as well as the age, sex, and weight of the patient, the severity of the symptoms, along with other factors.

The daily dosage of eribulin (e.g., eribulin mesylate) can be in the range of, e.g., 0.001 mg/m² to about 100 mg/m² (e.g., in the range of about 0.1 mg/m² to about 50 mg/m² or in the range of about 0.7 mg/m² to about 1.5 mg/m², or in any single amount within these ranges (e.g., 1.4 mg/m² or 1.1 mg/m²)). Eribulin can be administered as a single dose once a day, week, bi-week, month, or year, or more than one dose of eribulin can be administered per day, week, bi-week, month, or year. The administration can optionally be intravenous, e.g., for about 1 to about 20 minutes, or for about 2 to about 5 minutes. For example, in one administration protocol, eribulin can be administered once on days 1 and 8 of a 21-day cycle. More specifically, a recommended dose of eribulin (e.g., eribulin mesylate) is 1.4 mg/m² administered intravenously over 2 to 5 minutes on days 1 and 8 of a 21-day cycle. A recommended dose of eribulin (e.g., eribulin mesylate) in patients with mild hepatic impairment (Child-Pugh A) is 1.1 mg/m² administered intravenously over 2 to 5 minutes on days 1 and 8 of a 21-day cycle, while a recommended dose of eribulin (e.g., eribulin mesylate) in patients with moderate hepatic impairment (Child-Pugh B) is 0.7 mg/m² administered intravenously over 2 to 5 minutes on days 1 and 8 of a 21-day cycle. Further, a recommended dose of eribulin (e.g., eribulin mesylate) in patients with moderate renal impairment (creatinine clearance of 30-50 mL/min) is 1.1 mg/m² administered intravenously over 2 to 5 minutes on days 1 and 8 of a 21-day cycle. In another example, eribulin (e.g., eribulin mesylate) can be administered on a bi-weekly schedule, e.g., once on each of days 1 and 15 of a 28-day cycle. More specifically, an exemplary dose of eribulin (e.g., eribulin mesylate) is 1.4 mg/m² administered intravenously over 2 to 5 minutes on each of days 1 and 15 of a 28-day cycle. The dosage reductions noted above, 1.1 mg/m² in the case of patients with mild hepatic impairment or moderate renal impairment, and 0.7 mg/m² in the case of patients with moderate hepatic impairment, can also be used in bi-weekly regimens. These or other lower doses of eribulin (e.g., eribulin mesylate) can optionally be used in the context of patients having adverse reactions (e.g., hematologic or other adverse reactions) or in combination treatment, according to the methods of the present invention.

mTOR inhibitors can be administered using standard approaches and dosing regimens in the art. Everolimus, for example, can be orally administered in a range of about 1 mg/day to about 20 mg/day (e.g., 1, 2.5, 5, 7.5, 10, or 15 mg/day), in single or divided doses. Everolimus can be administered as a single dose once a day, week, month, or year, or more than one dose of everolimus can be administered per day, week, month, or year. For example, in one administration protocol, everolimus can be administered daily during a course of treatment with eribulin (see above) and, optionally, everolimus administration can continue beyond eribulin treatment regimens. In other embodiments, everolimus can be administered in decreasing step doses, such that the second and subsequent doses administered are reduced relative to the first, and preceding, dose. In the case of other examples of mTOR inhibitors, ridaforolimus (Merck & Company and Ariad Pharmaceuticals), for example, can be administered at 40 mg QDx5/week, while temsirolimus (Pfizer Inc.) can be administered in the amount of 25 mg infused one time per week over the course of 30-60 minutes. These amounts and regimens can be varied as determined to be appropriate by those of skill in the art.

Eribulin and mTOR inhibitor (e.g., everolimus) compositions can be administered to a patient substantially simultaneously or sequentially and in either order (e.g., administration of eribulin prior to the mTOR inhibitor (e.g., everolimus), or vice versa). In one example, eribulin is administered before (e.g., 1-12 hours or 1-3 days before) the start of mTOR inhibitor (e.g., everolimus) administration. Many regimens used to administer chemotherapeutic drugs involve, for example, intravenous administration of a drug (or drugs) followed by repetition of this treatment after a period (e.g., 1-4 weeks) during which the patient recovers from any adverse side effects of the treatment. It may be desirable to use both eribulin and an mTOR inhibitor (e.g., everolimus) at each administration or, alternatively, to have some (or all) of the treatments include only eribulin or only an mTOR inhibitor (e.g., everolimus).

As a specific, non-limiting example of a treatment regimen included in the invention, eribulin (e.g., 0.01-5 mg/m², e.g., 1.1 mg/m² or 1.4 mg/m²) is administered to a patient by intravenous infusion over 1 to 20 minutes (e.g., over 2 to 5 minutes) on days 1 and 8 of a 21-day cycle (or on days 1 and 15 of a 28-day cycle), while an mTOR inhibitor such as everolimus is administered daily (e.g., 1-20 mg or 10 mg) during this cycle. This course of treatment can be repeated (e.g., 1-8, 2-6, or 4-5 times), as determined to be tolerable and effective by those of skill in the art.

In addition to eribulin and an mTOR inhibitor (e.g., everolimus) the methods of the present invention can also include the administration of one or more additional therapeutic agents. Among these agents, immunomodulatory agents (e.g., antibodies or vaccines), chemotherapeutic/antitumor agents, antibacterial agents, anti-emetics, and anti-inflammatory agents are suitable. In one specific example, eribulin (e.g., eribulin mesylate) and an mTOR inhibitor (e.g., everolimus) are administered in combination with BKM-120 (Buparlisib), an orally bioavailable, specific oral inhibitor of the pan-class I phosphatidylinositol 3-kinase (PI3K) family of lipid kinases. In this example, the eribulin and mTOR inhibitor can optionally be administered as noted above, while the BKM-120 can be administered in the range of, e.g., about 0.01 mg to about 200 mg, e.g., about 50 mg to about 150 mg, or any single amount within this range (e.g., 100 mg), as a single dose once daily, weekly, or monthly during the course of, or beyond, the eribulin and mTOR treatment. In other instances, eribulin (e.g., eribulin mesylate) and an mTOR inhibitor (e.g., everolimus) can be used in a treatment regimen as the sole therapeutic (e.g., sole anti-cancer) agents. Thus, the methods of the invention can consist of administration of (i) eribulin or a pharmaceutically acceptable salt thereof (e.g., eribulin mesylate), and (ii) an mTOR inhibitor (e.g., everolimus).

The methods of the invention can be used to treat (including, e.g., delay progression) or prevent cancer in a subject (e.g., a human patient) and/or to decrease tumor size. The subject can be diagnosed with cancer, at risk for developing cancer, in treatment for cancer, or in post-therapy recovery from cancer. Further, the methods can be used to treat or prevent metastases and/or recurrence. The treatment can be chemotherapeutic alone, although treatment in combination with a surgical procedure to remove or reduce the size of a tumor, radiation therapy, immunotherapy, and/or ablation therapy is also envisioned.

Types of cancers that can be treated according to the present methods include, for example, breast cancer (e.g., estrogen receptor positive or negative, progesterone receptor positive or negative, HER-2 positive or negative, or triple-negative breast cancer), lung cancer (e.g., non-small cell lung cancer), ovarian cancer, pharyngeal cancer, esophageal cancer, and sarcoma.

### Kits

The invention also provides kits that include a container with eribulin (e.g., eribulin mesylate) and/or a container with mTOR inhibitor (e.g., everolimus). The eribulin (e.g., eribulin mesylate) and/or mTOR inhibitor (e.g., everolimus) in such kits can be provided in amounts sufficient to treat cancer in a patient in need thereof (e.g., amounts sufficient for a single administration or for multiple administrations). The kits can thus include multiple containers that each include effective amounts of single-dose eribulin (e.g., eribulin mesylate) and/or mTOR inhibitor (e.g., everolimus) pharmaceutical composition(s). Optionally, instruments and/or devices necessary for administering the pharmaceutical composition(s) can also be included in the kits. Furthermore, the kits can include additional components, such as instructions or administration schedules, for treating a patient with cancer with the eribulin (e.g., eribulin mesylate) and/or mTOR inhibitor (e.g., everolimus).

The present invention is illustrated by the following examples, which are in no way intended to be limiting of the invention.

### EXPERIMENTAL EXAMPLES

### Example 1

### Summary

The objective of this study was to determine the effect of E7389 (eribulin mesylate) when administered in combination with everolimus on the growth of subcutaneously-implanted human MX-1 mammary tumor xenografts in female athymic NCr-nu/nu mice. A total of 120 tumor bearing mice were divided into twelve groups of 10 mice. Group 1 was treated with E7389 and everolimus vehicles [2.5% DMSO/97.5% saline, intravenously (IV), once every four days for a total of four injections (Q4Dx4) and 0.5% methyl cellulose/0.2% polysorbate 80 in water for injection, oral gavage (PO), once daily for 16 consecutive days (Q1Dx16)], respectively. Groups 2, 3, and 4 were treated with E7389 at three doses (0.6, 0.4, and 0.2 mg/kg/injection) administered IV on a Q4Dx4 schedule. Groups 5 and 6 were treated with everolimus at two doses (40 and 20 mg/kg/dose) administered PO on a Q1Dx16 schedule. Groups 7, 8, and 9 were treated with E7389 at doses of 0.6, 0.4, or 0.2 mg/kg/injection in combination with everolimus at a dose 40 mg/kg/dose. Groups 10, 11, and 12 were treated with E7389 at doses of 0.6, 0.4, or 0.2 mg/kg/injection in combination with everolimus at a dose 20 mg/kg/dose. Everolimus was administered six hours after E7389. All treatments were initiated 10 days after the implantation of tumor xenografts. Growth of the MX-1 human mammary tumor xenografts was sensitive to the treatment with E7389 when administered IV on a Q4Dx4 schedule at doses of 0.6, 0.4, and 0.2 mg/kg/injection, resulting in statistically significant inhibition of the tumor growth in all three groups. Administration of E7389 at a dose of 0.6 mg/kg/injection produced six complete tumor regressions and five tumor-free survivors. Administration of everolimus PO at doses of 40 and 20 mg/kg/dose on a Q1Dx16 schedule resulted in minimal but statistically significant inhibition of the tumor growth at both doses tested. The treatments with E7389 at doses of 0.6, 0.4, and 0.2 mg/kg/injection in combination with everolimus at a dose of 40 or 20 mg/kg/dose were tolerated and resulted in statistically significant inhibition of the growth of the MX-1 mammary tumor xenografts.

Growth of the tumors in two combination groups in which E7389 was administered at a dose of 0.6 mg/kg/injection in combination with everolimus at a dose of 40 or 20 mg/kg/dose were not statistically different from the growth of the tumors in the groups treated with E7389 alone based on individual animals' times to reach four tumor mass doublings and individual animals' tumor weights on Day 62. While growth of the tumors in the groups in which E7389 was administered at a dose of 0.4 mg/kg/injection in combination with everolimus at a dose of 40 or 20 mg/kg/dose was statistically different from the growth of the tumors in the group treated with E7389 alone when individual animals' times to reach four tumor mass doublings were compared, the difference did not reach significance when individual animals' tumor weights on Day 52 were compared. The antitumor activity of both combination treatments was greater than additive compared to the antitumor activity produced by administration of each compound alone based on comparison of median tumor growth delays. Growth of the tumors in two groups in which E7389 was administered at a dose of 0.2 mg/kg/injection in combination with everolimus at a dose of 40 or 20 mg/kg/dose was statistically different from the growth of the tumors in the group treated with E7389 alone when individual animals' times to reach four tumor mass doublings and individual animals' tumor weights on Day 41 were compared. The antitumor activity of both combination treatments was additive compared to the antitumor activity produced by administration of each compound alone based on comparison of median tumor growth delays. Administration of everolimus resulted in temporary dryness of the skin of the animals (such dryness of the skin was not noted in the control or E7389-treated groups). The severity of the skin dryness in all combination groups appeared to be less than seen in the groups treated with everolimus alone. The antitumor efficacy of compound E7389 when administered in combination with a mTOR (mammalian target of rapamycin) inhibitor, everolimus, to female athymic NCr-nu/nu mice implanted subcutaneously (SC) with MX-1 human mammary tumor xenografts was evaluated in this study.

### Materials and Methods

### Animal Care

Six-weeks-old female, athymic NCr-nu/nu mice were purchased from Charles River Laboratories (Wilmington, Mass.) and acclimated in the laboratories for 10 days prior to experimentation. The animals were housed in microisolator cages, five per cage with a 12-hour light/dark cycle. The animals received filtered Birmingham municipal water and sterilized Teklad Global 16% protein rodent diet (2016S, Harlan Laboratories, Inc.) ad libitum. No consumable enrichment was provided. Enrich-n'Nest paper rolls (the Andersons Lab Bedding Products, Maumee, Ohio) were provided in each cage as manipulanda. Cages were changed twice weekly. The animals were observed daily and clinical signs were noted.

### Tumor Model

Each mouse was implanted SC near the right flank with a 30-40 mg fragment of MX-1 human mammary tumor from an in vivo passage using a 13-gauge needle. The day of tumor fragments implantation was designated as Day 0. Individual tumors of 120 animals grew to 100-245 mg in weight (100-245 mm³ in size) on the day of treatment initiation, Day 10 after tumor fragments implantation. Those animals selected with tumors in the proper size range were assigned to twelve treatment groups so that the mean and median tumor weights in all groups on the first day of treatment were as close to each other as possible (mean tumor weights ranged from 160 to 168 mg, median tumor weights were 153 or 162 mg).

### Drug Storage

A vial with E7389 powder (eribulin mesylate, 10.1 mg) was received from Eisai Inc. frozen (shipped on dry ice) and was stored at below −70° C. in the dark on desiccant upon receipt. Everolimus (>99%, catalog no. E4040) was purchased from LC Laboratories and was stored at −20° C. upon receipt. DriSolv® methyl sulfoxide (DMSO, anhydrous, catalog no. MX 1457-7) was purchased from EMD Chemicals, Inc. and was stored at room temperature upon receipt. Once the bottle with DMSO was opened it was stored at room temperature under nitrogen. Saline (physiological saline solution, sterile, preservative free, for animal use only) and Water for Injection, USP (WFI, sterile—nonpyrogenic, for animal use only) were manufactured by Nova-Tech, Inc. and were stored at room temperature. During the formulation period, saline and WFI were stored at 4° C. Methyl cellulose (MC, viscosity of a 2% aqueous solution at 20° C. of 4,000 cP) was purchased from Sigma-Aldrich and was stored at room temperature. Polysorbate 80 (T80, Fisher Scientific) was stored at room temperature. The solution of 0.5% MC/0.2% T80 in WFI was stored at 4° C.

### Drug Formulation

A 2.4 mg/mL stock solution of E7389 in 100% DMSO was formulated by adding 4.21 mL of 100% DMSO (from an unopened bottle) to the vial with 10.1 mg of E7389 and dissolving E7389 in 100% DMSO by gentle vortexing. The solution was allowed to stand for 2-3 minutes to make sure it was fully in solution. The 2.4 mg/mL stock solution was aliquoted for all 4 days of treatment, the air in the vials with aliquots and the remaining 2.4 mg/mL stock was displaced with nitrogen, and the vials with the aliquots and the remaining stock were frozen at below −70° C. On each day of treatment, an aliquot was thawed at room temperature and diluted with saline to yield a concentration of 0.06 mg/mL in 2.5% DMSO/97.5% saline. Lower concentrations of 0.04 and 0.02 mg/mL were achieved by dilution of a portion of the 0.06 mg/mL solution with 2.5% DMSO/97.5% saline. Bottles with dosing solutions were stored at 4° C. between formulation and administration.

100% DMSO was aliquoted for each day of treatment, the air in the vials was displaced with nitrogen, and the vials were frozen at below −70° C. On each day of treatment an aliquot of the DMSO was thawed at room temperature and diluted with saline to yield 2.5% DMSO/97.5% saline. This solution was used to treat mice in Group 1 (E7389 vehicle).

0.5% MC/0.2% T80 in WFI was formulated and the solution was stored at 4° C. Everolimus at a concentration of 4 mg/mL was formulated on each day of treatment in 0.5% MC/O.2% T80 in WFI. A portion of the 4 mg/mL cloudy solution was diluted with 0.5% MC/0.2% T80 in WFI to 2 mg/mL. Bottles with dosing solutions were stored at 4° C. between formulation and administration.

All E7389 dosing solutions, everolimus dosing solutions, and the vehicles were administered to mice by exact individual body weight on each day of treatment, with the injection volume being 0.1 mL/10 g body weight.

### Drug Treatment

The experiment consisted of a vehicles-treated control group and eleven drug-treated groups of 10 mice per group for a total of 120 mice on the first day of treatment. All treatments were initiated on Day 10. E7389 was administered intravenously (IV) once every four days for a total of four injections (Q4Dx4; Days 10, 14, 18, and 22, also referred to as Q4Dx4(10)-(0) in all Figures) at doses of 0.6, 0.4, and 0.2 mg/kg/injection alone (Groups 2-4, respectively), and in combination with everolimus (Groups 7-12). Everolimus was administered by oral gavage (PO) once daily for 16 consecutive days (QIDxI6; Days 10-25, also referred to as Q1Dx16(10)-(6) in all Figures) at doses of 40 and 20 mg/kg/dose alone (Groups 5 and 6, respectively) and in combination with E7389 (Groups 7-12). On the days when both compounds were administered, E7389 was administered to all combination groups first [−(0)] (injections were initiated at 10:05, 8:45, 8:40, and 8:32 a.m. on Days 10, 14, 18, and 22, respectively) followed by the administration of everolimus six hours later [−(6)]. On the days when only everolimus was administered the treatment was performed in the second part of the day (between 1:15 and 2:45 pm) except on Day 15, when the treatment was started at 11:11 am. The control group (Group 1) was treated with E7389 and everolimus vehicles (2.5% DMSO/97.5% saline, IV, Q4Dx4 and 0.5% MC/0.2% T80 in WFI, PO, Q1Dx16, respectively).

### Tumor Measurements and Body Weights

Animals were observed once daily for mortality and morbidity. The SC tumors were measured and the animals were weighed twice weekly starting with the first day of treatment, Day 10 after tumor fragment implantation. Tumor volume was determined by caliper measurements (mm) and using the formula for an ellipsoid sphere: I×w²/2=mm³ where I and w refer to the larger and smaller perpendicular dimensions collected at each measurement. This formula was also used to calculate tumor weight, assuming unit density (1 mm3=1 mg). Limit of tumor detection was 4×4 mm (32 mg).

### Study Duration

The study was terminated on Day 62 after tumor fragment implantation. Any animal whose tumor reached 4,000 mg in weight or ulcerated or any animal whose body weight decreased below 14 g was euthanized prior to the scheduled day of study termination for humane reasons.

### Parameters Evaluated

Number of nonspecific deaths, number of complete tumor regressions, number of tumor-free survivors on Day 62, and median number of days for the tumors in each group to reach four tumor mass doublings were calculated. The median time to reach four tumor mass doublings was used in the calculation of the overall delay in the growth of the median tumor (T-C, days). Comparison of the median tumor weight in the treatment groups (T) to the median tumor weight in the control group (T/C×100%) on Day 34 (the last day of data collection when more than 50% of the animals were still alive in the control group) was used for an additional evaluation of the antitumor efficacy. Additionally, change in group mean body weight on each day of data collection relative to the group mean body weight on Day 10 (in grams and as a percent) was calculated for each group.

### Data Analysis

Individual tumor measurements and body weights were collected and processed using the software ADAS (Automated Data Acquisition System) developed at Southern Research Institute. SigmaPlot™ 9.0 software was used to present graphically the body and tumor weight data. SigmaStat version 3.5 statistical software was used to compare statistically the tumor growth data. The difference between the groups was considered to be significant if the P value was equal to or less than 0.05. The individual animal's time to reach four tumor mass doublings was used as the endpoint in a life tables analysis (Kaplan-Meier survival analysis followed by a log-rank test). The life tables analysis allows for the comparison of the growth data between the groups using the animals whose tumors did not reach the evaluation point, by censoring them. The individual animals' tumor weights on Day 62 for three groups treated with E7389 at a dose of 0.6 mg/kg/injection (alone or in combination with everolimus), on Day 52 for three groups treated with E7389 at a dose of 0.4 mg/kg/injection, and on Day 41 for three groups treated with E7389 at a dose of 0.2 mg/kg/injection were compared by t-test (or Mann-Whitney rank sum test). Nonparametric test was used when the data set did not the pass the normality test. The day selected for analysis was the last day of data collection when at least 50% of the animals were alive in all three groups.

### Results

Tumors in the vehicles-treated control group (Group 1) grew well in all ten mice. One tumor ulcerated before reaching four tumor mass doublings. Median tumor reached four tumor mass doublings in 18.2 days and reached 4,513 mg in weight on Day 34. Animals gained weight over the course of the experiment.

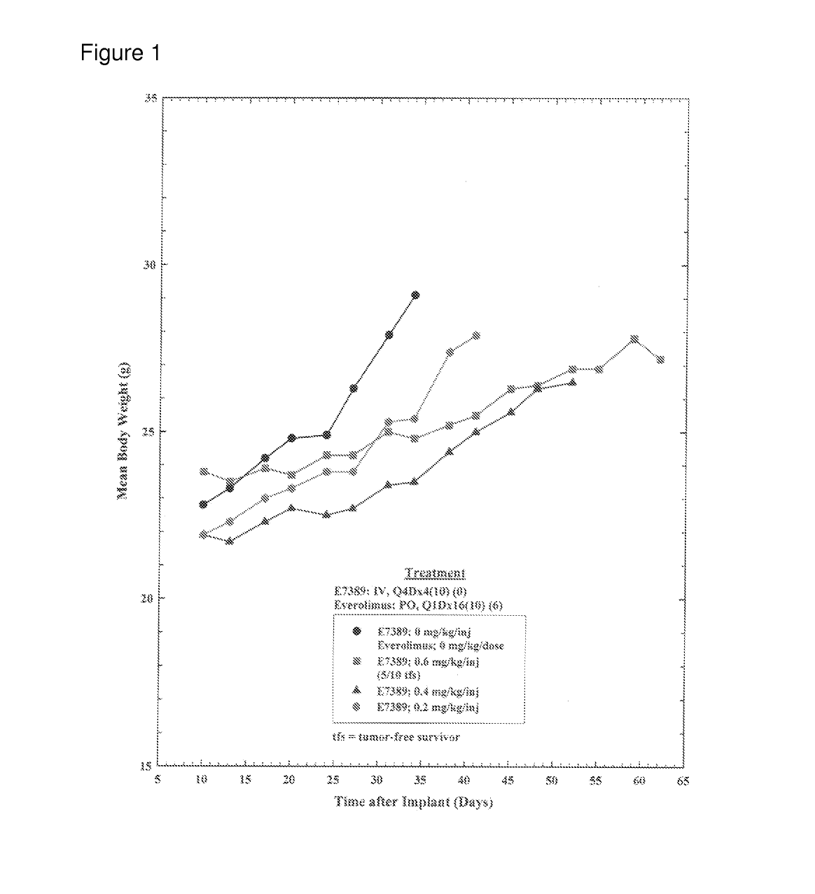

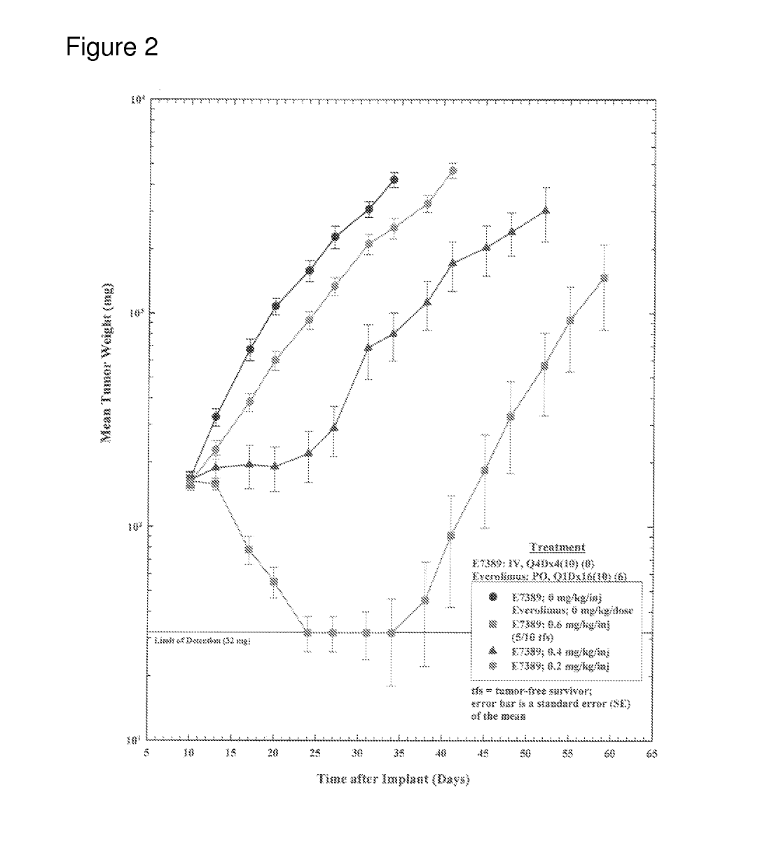

Intravenous administration of E7389 at doses of 0.6, 0.4, and 0.2 mg/kg/injection on a Q4Dx4 schedule (Groups 2, 3, and 4, respectively) was tolerated without treatment-related deaths. The treatment was associated with a maximum mean body weight loss of 1% (0.2-0.3 g), when E7389 was administered at doses of 0.6 and 0.4 mg/kg/injection, respectively, while administration of the dose of 0.2 mg/kg/injection did not result in mean body weight loss. Thus, the maximum tolerated dose (MTD, defined as the dose which does not result in death of more than 10% of animals in the group or produces no more than 20% mean body weight loss) of E7389 when administered IV on a Q4Dx4 schedule was not reached in this experiment. The IV treatment with E7389 at doses of 0.6, 0.4, and 0.2 mg/kg/injection was very effective in the inhibition of the growth of the MX-1 mammary tumor xenografts. Tumors of six out of ten animals in the group treated with a dose of 0.6 mg/kg/injection underwent complete regression and five animals were tumor-free on the day of study termination. The median tumor growth delays in groups treated with doses of 0.6, 0.4, and 0.2 mg/kg/injection were >33.8, 17.7, and >6.3 days, respectively. The T/C values on Day 34 were 0%, 13%, and 51%, respectively. Growth of the tumors in the groups treated with E7389 at doses of 0.6, 0.4, and 0.2 mg/kg/injection was found to be statistically different from the growth of the tumors in the control group, when individual animal's times to reach four tumor mass doublings were compared (Group 1 vs. Group 2: P<0.001, Group 1 vs. Group 3: P<0.001, Group 1 vs. Group 4: P=0.001). Change in mean body weights of the animals treated with E7389 over the course of the experiment is presented graphically in FIG. 1. Response of the SC-implanted human MX-1 mammary tumor fragments to the treatment with E7389 is presented graphically in FIG. 2 (mean tumor weights).

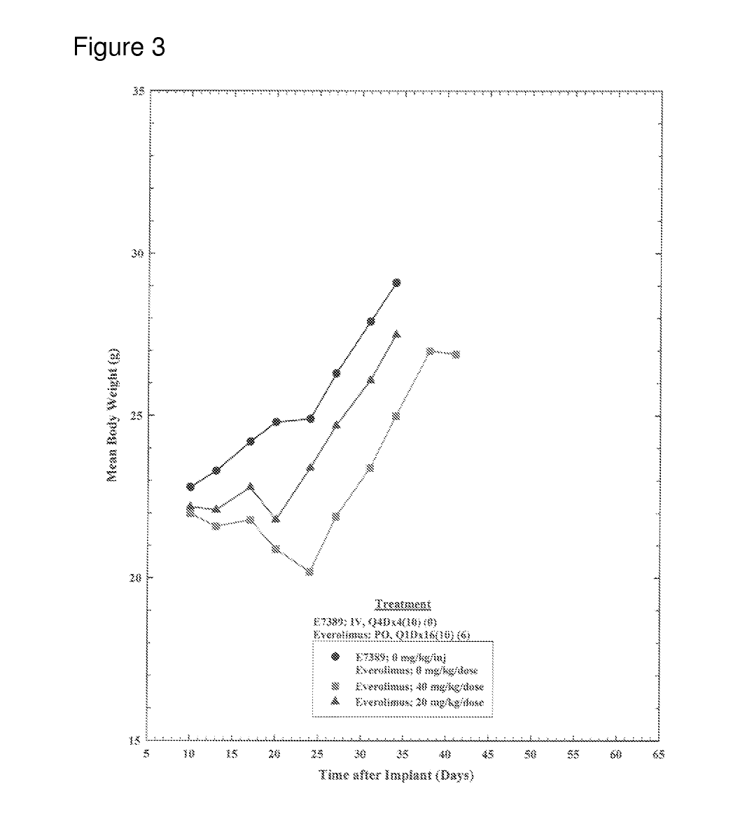

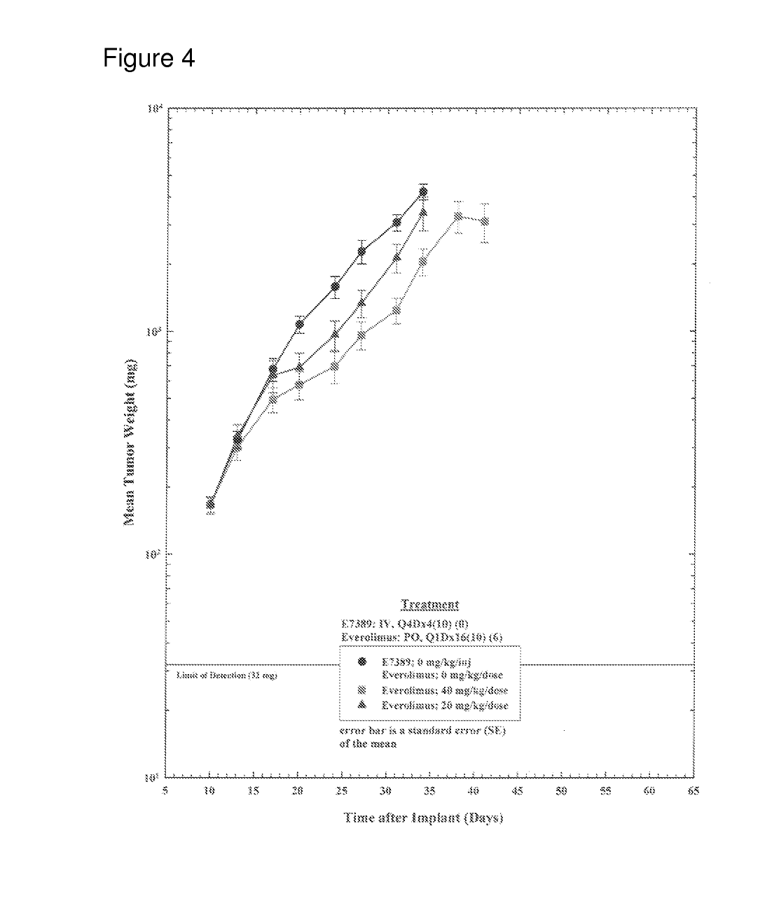

Oral administration of everolimus at doses of 40 and 20 mg/kg/dose on a Q1Dx16 schedule (Groups 5 and 6, respectively) was tolerated without treatment-related deaths. The treatment with a dose of 40 mg/kg/dose was associated with a maximum mean body weight loss of 8% (1.8 g), observed on Day 24. Four animals in the group treated with a dose of 20 mg/kg/dose died on Day 21. These deaths resulted from a flooded cage the night before (these animals were excluded from all tumor growth calculations). The treatment with a dose of 20 mg/kg/dose was associated with a maximum mean body weight loss of 2% (0.4 g), observed on Day 20. Thus, the MTD of everolimus when administered PO on a Q1Dx16 schedule was not reached in this experiment. All animals in both groups were noted to have dry skin starting on Day 20. By Day 31 the skin appeared normal. The PO treatment with everolimus at both doses tested was minimally effective in the inhibition of the growth of the MX-1 mammary tumor xenografts, producing median tumor growth delays of 6.1 and 4.0 days, respectively. The T/C values on Day 34 were 46% and 74%, respectively. Growth of the tumors in the groups treated with everolimus at doses of 40 and 20 mg/kg/dose was found to be statistically different from the growth of the tumors in the control group, when individual animal's time to reach four tumor mass doublings were compared (Group 1 vs. Group 5: P<0.001, Group 1 vs. Group 6: P=0.019). Change in mean body weights of the animals treated with everolimus over the course of the experiment is presented graphically in FIG. 3. Response of the SC-implanted human MX-1 mammary tumor fragments to the treatment with everolimus is presented graphically in FIG. 4 (mean tumor weights).

Administration of E7389 IV on a Q4Dx4 schedule at doses of 0.6, 0.4, and 0.2 mg/kg/injection plus everolimus PO on a Q1Dx16 schedule at a dose of 40 mg/kg/dose (Groups 7, 8, and 9, respectively) was tolerated without deaths. One animal in the group treated with E7389 at a dose of 0.6 mg/kg/injection plus everolimus at a dose of 40 mg/kg/dose was euthanized on Day 24 due to excessive body weight loss. This was considered to be a treatment-related euthanasia. The combination treatments were associated with maximum mean body weight losses of 9% (2.1 g), 5% (1.2 g), and 8% (1.8 g), when E7389 was administered at doses of 0.6, 0.4, and 0.2 mg/kg/injection, respectively. Thus, the MTD of E7389 when administered IV on a Q4Dx4 schedule in combination with everolimus administered PO on a Q1Dx16 schedule was not reached in this experiment. Animals in all three groups were noted to have dry skin starting on Day 20. The severity of the skin dryness appeared to be less than seen in Group 5 (everolimus at a dose of 40 mg/kg/dose alone). By Day 27 the skin condition improved. The combination treatments exhibited dose-dependent antitumor activity, producing median tumor growth delays of >33.8, 30.0, and 13.4 days in the groups in which E7389 was administered at doses of 0.6, 0.4, and 0.2 mg/kg/injection, respectively. Tumors of nine and four animals in the groups in which E7389 was administered at doses of 0.6 and 0.4 mg/kg/injection, respectively, underwent complete regression and four and two animals, respectively, were tumor-free on the day of study termination. The T/C values in the combination groups in which E7389 was administered at doses of 0.6, 0.4, and 0.2 mg/kg/injection on Day 34 were 0%, 2%, and 19%, respectively. Growth of the tumors in all three combination groups was statistically different from the growth of the tumors in the control group (P<0.001 for all three groups), when individual animal's time to reach four tumor mass doublings were compared.

Growth of the tumors in the combination group in which E7389 was administered at a dose of 0.6 mg/kg/injection was not statistically different from the growth of the tumors in the group treated with E7389 alone when individual animals' times to reach four tumor mass doublings (Group 2 vs. Group 7: P=0.285) and individual animals' tumor weights on Day 62 (Group 2 vs. Group 7: P=0.567) were compared. While growth of the tumors in the combination group in which E7389 was administered at a dose of 0.4 mg/kg/injection was statistically different from the growth of the tumors in the group treated with E7389 alone when individual animals' times to reach four tumor mass doublings were compared (Group 3 vs. Group 8: P=0.032), the difference did not reach significance when individual animals' tumor weights on Day 52 were compared (Group 3 vs. Group 8: P=0.083). The antitumor activity of this combination treatment was greater than additive compared to the antitumor activity produced by administration of each compound alone: a median tumor growth delay in Group 3 (E7389 at a dose of 0.4 mg/kg/injection alone)=17.7 days, a median tumor growth delay in Group 5 (everolimus at a dose of 40 mg/kg/dose alone)=6.1 days compared to a median tumor growth delay in Group 8 (E7389 at a dose of 0.4 mg/kg/injection plus everolimus at a dose of 40 mg/kg/dose)=30.0 days. Growth of the tumors in the combination group in which E7389 was administered at a dose of 0.2 mg/kg/injection was statistically different from the growth of the tumors in the group treated with E7389 alone when individual animals' times to reach four tumor mass doublings (Group 4 vs. Group 9: P<0.001) and individual animals' tumor weights on Day 41 (Group 4 vs. Group 9: P<0.001) were compared. The antitumor activity of the combination treatment was additive compared to the antitumor activity produced by administration of each compound alone: a median tumor growth delay in Group 4 (E7389 at a dose of 0.2 mg/kg/injection alone)=>6.3 days, a median tumor growth delay in Group 5 (everolimus at a dose of 40 mg/kg/dose alone)=6.1 days compared to a median tumor growth delay in Group 9 (E7389 at a dose of 0.2 mg/kg/injection plus everolimus at a dose of 40 mg/kg/dose)=13.4 days.

Administration of E7389 IV on a Q4Dx4 schedule at doses of 0.6, 0.4, and 0.2 mg/kg/injection plus everolimus PO on a Q1Dx16 schedule at a dose of 20 mg/kg/dose (Groups 10, 11, and 12, respectively) was tolerated without deaths. The combination treatments were associated with maximum mean body weight losses of 1% (0.3 g), 2% (0.4 g), and 3% (0.8 g), when E7389 was administered at doses of 0.6, 0.4, and 0.2 mg/kg/injection, respectively. Animals in all three groups were noted to have dry skin starting on Day 20. The severity of the skin dryness appeared to be less than seen in Group 6 (everolimus at a dose of 20 mg/kg/dose alone). The combination treatments exhibited dose-dependent antitumor activity, producing median tumor growth delays of >33.8, 25.1, and 12.9 days in the groups in which E7389 was administered at doses of 0.6, 0.4, and 0.2 mg/kg/injection, respectively. Tumors of eight and one animals in the groups in which E7389 was administered at doses of 0.6 and 0.4 mg/kg/injection, respectively, underwent complete regression and one animal in each group was tumor-free on the day of study termination. The T/C values in the combination groups in which E7389 was administered at doses of 0.6, 0.4, and 0.2 mg/kg/injection on Day 34 were 0%, 3%, and 23%, respectively. Growth of the tumors in all three combination groups was statistically different from the growth of the tumors in the control group (P<0.001 for all three groups), when individual animal's time to reach four tumor mass doublings were compared.

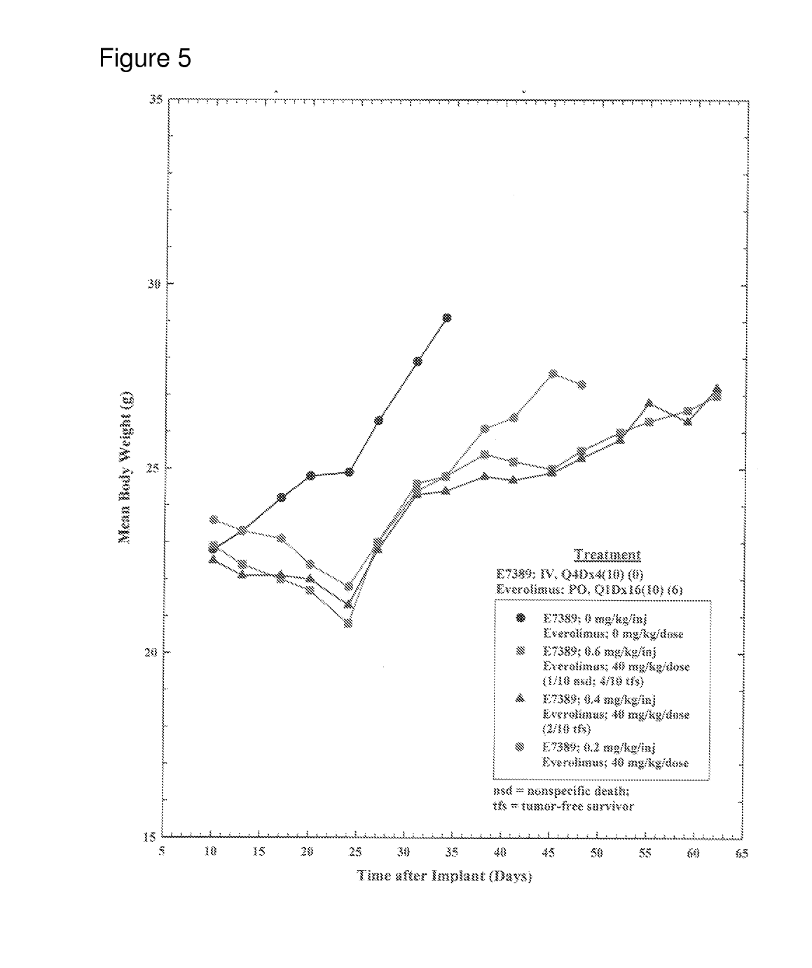

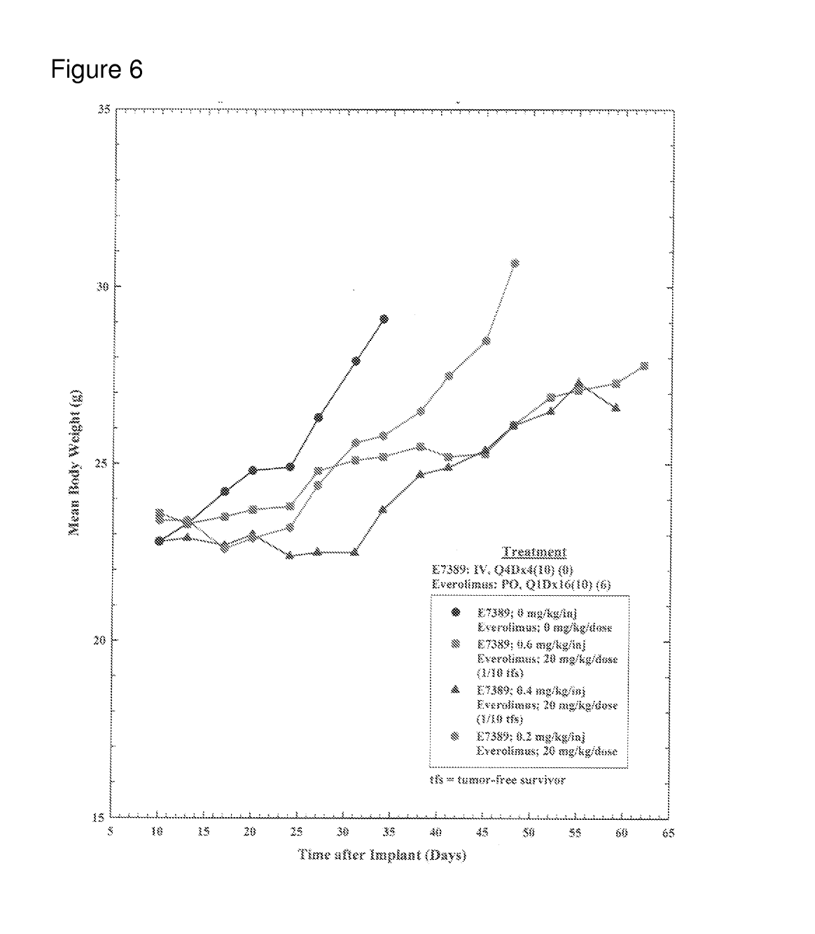

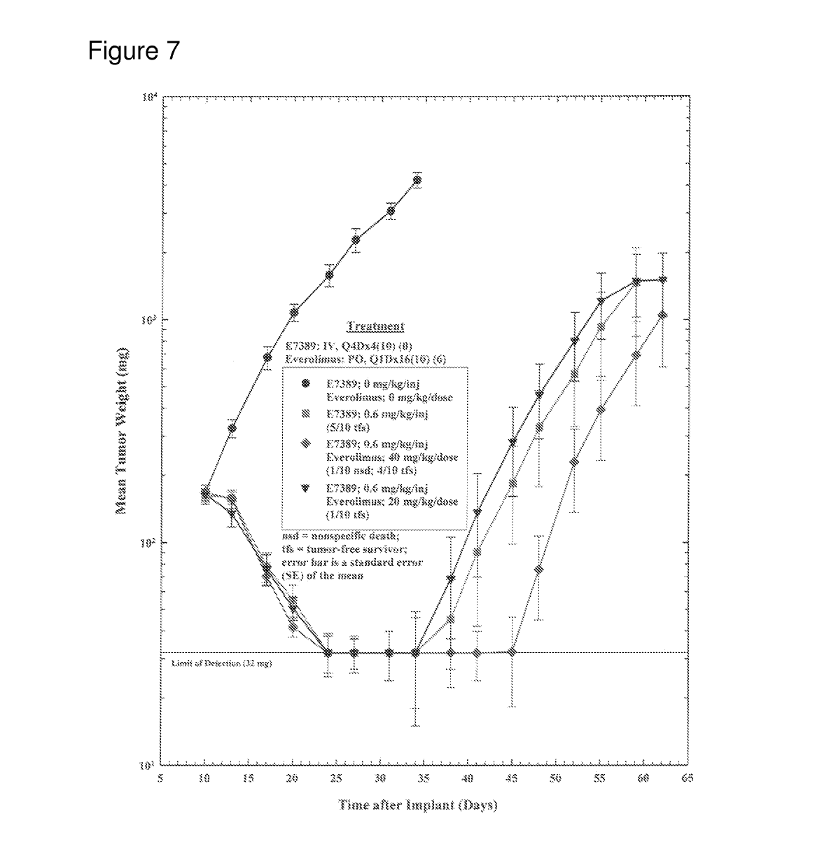

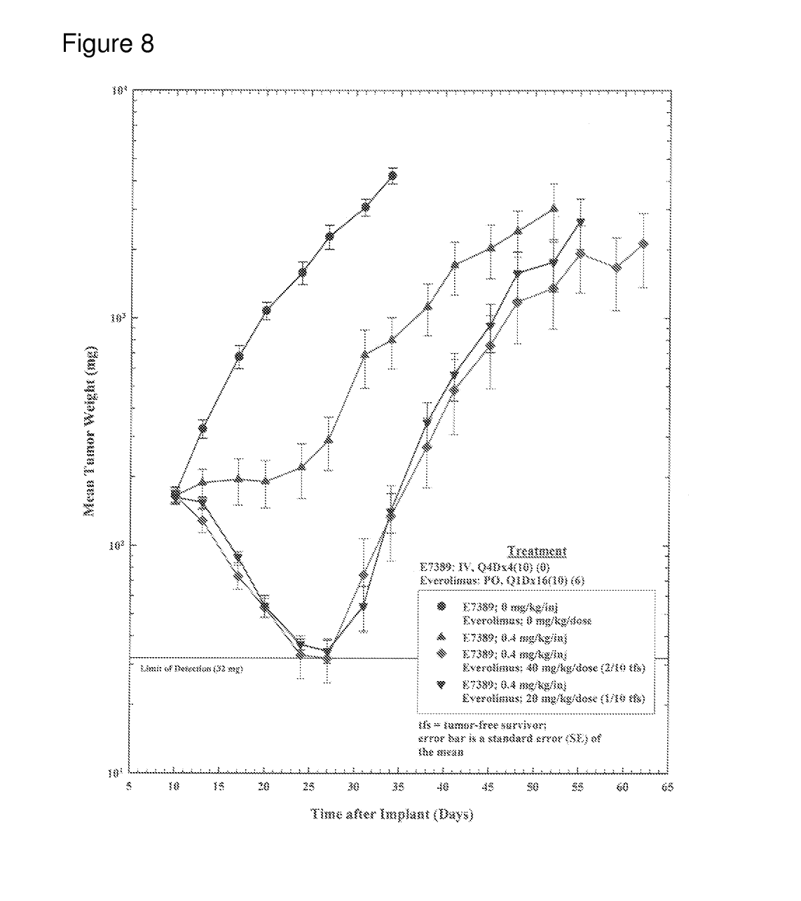

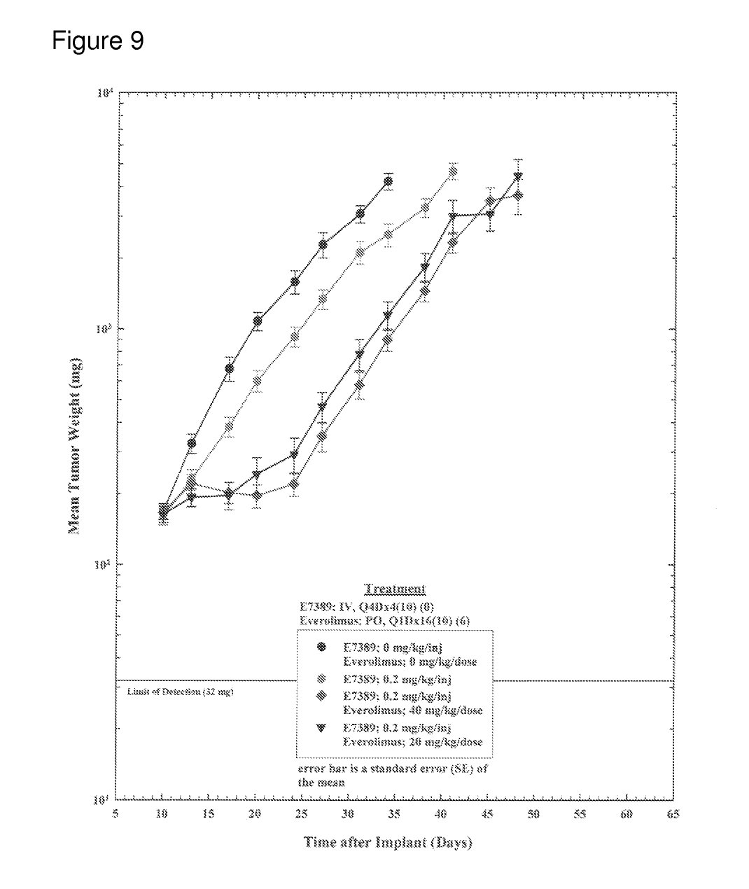

Growth of the tumors in the combination group in which E7389 was administered at a dose of 0.6 mg/kg/injection was not statistically different from the growth of the tumors in the group treated with E7389 alone when individual animals' times to reach four tumor mass doublings (Group 2 vs. Group 10: P=0.985) and individual animals' tumor weights on Day 62 (Group 2 vs. Group 10: P=0.161) were compared. While growth of the tumors in the combination group in which E7389 was administered at a dose of 0.4 mg/kg/injection was statistically different from the growth of the tumors in the group treated with E7389 alone when individual animals' times to reach four tumor mass doublings were compared (Group 3 vs. Group 11: P=0.05), the difference did not reach significance when individual animals' tumor weights on Day 52 were compared (Group 3 vs. Group 11: P=0.180). The antitumor activity of this combination treatment was greater than additive compared to the antitumor activity produced by administration of each compound alone: a median tumor growth delay in Group 3 (E7389 at a dose of 0.4 mg/kg/injection alone)=17.7 days, a median tumor growth delay in Group 6 (everolimus at a dose of 20 mg/kg/dose alone)=4.0 days compared to a median tumor growth delay in Group 11 (E7389 at a dose of 0.4 mg/kg/injection plus everolimus at a dose of 20 mg/kg/dose)=25.1 days. Growth of the tumors in the combination group in which E7389 was administered at a dose of 0.2 mg/kg/injection was statistically different from the growth of the tumors in the group treated with E7389 alone when individual animals' times to reach four tumor mass doublings (Group 4 vs. Group 12: P<0.001) and individual animals' tumor weights on Day 41 (Group 4 vs. Group 9: P=0.029) were compared. The antitumor activity of this combination treatment was additive compared to the antitumor activity produced by administration of each compound alone: a median tumor growth delay in Group 4 (E7389 at a dose of 0.2 mg/kg/injection alone)=>6.3 days, a median tumor growth delay in Group 6 (everolimus at a dose of 20 mg/kg/dose alone)=4.0 days compared to a median tumor growth delay in Group 12 (E7389 at a dose of 0.2 mg/kg/injection plus everolimus at a dose of 20 mg/kg/dose)=12.9 days. Change in mean body weights of the animals treated with E7389 at three doses in combination with everolimus at a dose of 40 or 20 mg/kg/dose over the course of the experiment is presented graphically in FIG. 5 and FIG. 6, respectively. Response of the SC-implanted human MX-1 mammary tumor fragments to the treatment with E7389 at a dose of 0.6, 0.4 or 0.2 mg/kg/injection in combination with two doses of everolimus is presented graphically in FIG. 7, FIG. 8, and FIG. 9, respectively (mean tumor weights).

### CONCLUSIONS

Growth of the MX-1 human mammary tumor xenografts was sensitive to the treatment with E7389 when administered IV on a Q4Dx4 schedule at doses of 0.6, 0.4, and 0.2 mg/kg/injection, resulting in statistically significant inhibition of the tumor growth in all three groups. Administration of E7389 at a dose of 0.6 mg/kg/injection produced six complete tumor regressions and five tumor-free survivors. Administration of everolimus PO at doses of 40 and 20 mg/kg/dose on a Q1Dx16 schedule resulted in minimal but statistically significant inhibition of the tumor growth at both doses tested. The treatments with E7389 at doses of 0.6, 0.4, and 0.2 mg/kg/injection in combination with everolimus at a dose of 40 or 20 mg/kg/dose were tolerated and resulted in statistically significant inhibition of the growth of the MX-1 mammary tumor xenografts.

Growth of the tumors in two combination groups in which E7389 was administered at a dose of 0.6 mg/kg/injection in combination with everolimus at a dose of 40 or 20 mg/kg/dose were not statistically different from the growth of the tumors in the groups treated with E7389 alone based on individual animals' times to reach four tumor mass doublings and individual animals' tumor weights on Day 62. While growth of the tumors in the groups in which E7389 was administered at a dose of 0.4 mg/kg/injection in combination with everolimus at a dose of 40 or 20 mg/kg/dose was statistically different from the growth of the tumors in the group treated with E7389 alone when individual animals' times to reach four tumor mass doublings were compared, the difference did not reach significance when individual animals' tumor weights on Day 52 were compared. The antitumor activity of both combination treatments was greater than additive compared to the antitumor activity produced by administration of each compound alone. Growth of the tumors in two groups in which E7389 was administered at a dose of 0.2 mg/kg/injection in combination with everolimus at a dose of 40 or 20 mg/kg/dose was statistically different from the growth of the tumors in the group treated with E7389 alone when individual animals' times to reach four tumor mass doublings and individual animals' tumor weights on Day 41 were compared. The antitumor activity of both combination treatments was additive compared to the antitumor activity produced by administration of each compound alone.

Administration of everolimus resulted in temporary dryness of the skin of the animals (such dryness of the skin was not noted in the control or E7389-treated groups). The severity of the skin dryness in all combination groups appeared to be less than seen in the groups treated with everolimus alone.

### Example 2

### Methods

### Anti-Proliferation Assay

The methods by which the high throughput screens for the experiments described in this example were carried out are described below.

Cells are thawed from a liquid nitrogen preserved state. Once cells have been expanded and divide at their expected doubling times, screening begins. Cells are seeded in growth media in 384-well and 1536-well tissue culture treated plates. Cells are equilibrated in assay plates via centrifugation and placed in incubators attached to dosing modules at 37° C. for twenty-four hours before treatment. At the time of treatment, a set of assay plates (which do not receive treatment) is collected and ATP levels are measured by adding ATPLite (Perkin Elmer). These Tzero (T0) plates are read using ultra-sensitive luminescence on Envision Plate Readers. Treated assay plates are incubated with compound for seventy-two hours. After seventy-two hours, plates are developed for endpoint analysis using ATPLite. All data points are collected via automated processes, quality controlled, and analyzed using software. Assay plates are accepted if they pass the following quality control standards: relative luciferase values are consistent throughout the entire experiment, Z-factor scores are greater than 0.6, and untreated/vehicle controls behave consistently on the plate. The calculation for synergy score is provided below.

Growth Inhibition (GI) is used as a measure of cell viability. The cell viability of vehicle is measured at the time of dosing (T0) and after seventy-two hours (T72). A GI reading of 0% represents no growth inhibition, i.e., cells treated with compound and T72 vehicle signals are matched. A GI 100% represents complete growth inhibition, i.e., cells treated by compound and T0 vehicle signals are matched. Cell numbers have not increased during the treatment period in wells with GI 100% and may suggest a cytostatic effect for compounds reaching a plateau at this effect level. A GI 200% represents complete death of all cells in the culture well. Compounds reaching an activity plateau of GI 200% are considered cytotoxic. GI is calculated by applying the following test and equation:

$$If T < {V}_{0} \text{:} 100 * ( 1 - \frac{T - {V}_{0}}{{V}_{0}} )$$

$$If T \geq {V}_{0} \text{:} 100 * ( 1 - \frac{T - {V}_{0}}{V - {V}_{0}} )$$

where T is the signal measure for a test article, V is the vehicle-treated control measure, and Vo is the vehicle control measure at time zero. This formula is derived from the Growth Inhibition calculation used in the National Cancer Institute's NCI-60 high throughput screen. All single agent and combination data analysis are performed in growth inhibition.

### Synergy Score Analysis

To measure combination effects in excess of Loewe additivity, a scalar measure is used to characterize the strength of synergistic interaction termed the Synergy Score. The Synergy Score is calculated as:

Synergy Score=log fₓ log f_{y}Σmax(0,I_{data}(I_{data}−I_{Loewe})

The fractional inhibition for each component agent and combination point in the matrix is calculated relative to the median of all vehicle-treated control wells. The Synergy Score equation integrates the experimentally-observed activity volume at each point in the matrix in excess of a model surface numerically derived from the activity of the component agents using the Loewe model for additivity. Additional terms in the Synergy Score equation (above) are used to normalize for various dilution factors used for individual agents and to allow for comparison of synergy scores across an entire experiment. The inclusion of positive inhibition gating or an Idata multiplier removes noise near the zero effect level, and biases results for synergistic interactions at that occur at high activity levels.

Potency shifting is evaluated using an isobologram, which demonstrates how much less drug is required in combination to achieve a desired effect level when compared to the single agent doses needed to reach that effect. An isobologram is drawn by identifying the locus of concentrations that correspond to crossing the indicated inhibition level. This is done by finding the crossing point for each single agent concentration in a dose matrix across the concentrations of the other single agent. Practically, each vertical concentration CY is held fixed while a bisection algorithm is used to identify the horizontal concentration CX in combination with that vertical dose that gives the chosen effect level in the response surface Z(CX,CY). These concentrations are then connected by linear interpolation to generate the isobologram display. For synergistic interactions, the isobologram contour fall below the additivity threshold and approaches the origin, and an antagonistic interaction would lie above the additivity threshold. The error bars represent the uncertainty arising from the individual data points used to generate the isobologram. The uncertainty for each crossing point is estimated from the response errors using bisection to find the concentrations where Z−σZ(CX,CY) and Z+σZ(CX,CY) cross Icut, where σZ is the standard deviation of the residual error on the effect scale.

### Loewe Volume Score Analysis

Loewe Volume is used to assess the overall magnitude of the combination interaction in excess of the Loewe additivity model. Loewe Volume is particularly useful when distinguishing synergistic increases in a phenotypic activity (positive Loewe Volume) versus synergistic antagonisms (negative Loewe Volume). When antagonisms are observed, the Loewe Volume should be assessed to examine if there is any correlation between antagonism and a particular drug target-activity or cellular genotype. This model defines additivity as a non-synergistic combination interaction where the combination dose matrix surface should be indistinguishable from either drug crossed with itself.

The calculation for additivity is:

I_{Loewe} that satisfies (X/X_{I})+(Y/Y_{I})=1

where XI and YI are the single agent effective concentrations for the observed combination effect I. For example, if 50% inhibition is achieved separately by 1 μM of drug A or 1 μM of drug B, a combination of 0.5 μM of A and 0.5 μM of B should also inhibit by 50%.

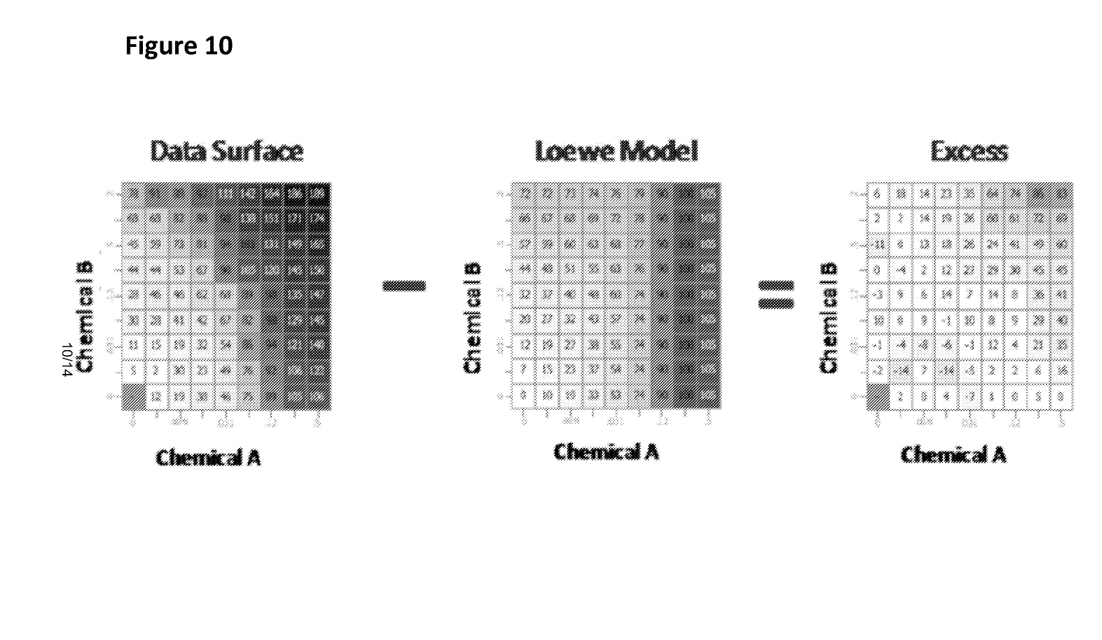

Activity observed in excess of Loewe additivity identifies potential synergistic interaction. For the present analysis, empirically derived combination matrices were compared to their respective Loewe additivity models constructed from experimentally collected single agent dose response curves. Any activity observed after subtraction of the additivity model from the dose response matrix is indicative of synergy (FIG. 10). Negative Loewe Volume is indicative of antagonism. Summation of this excess additivity across the dose response matrix is referred to as Loewe Volume.

### Results

### Single Agent Dose Response Analysis

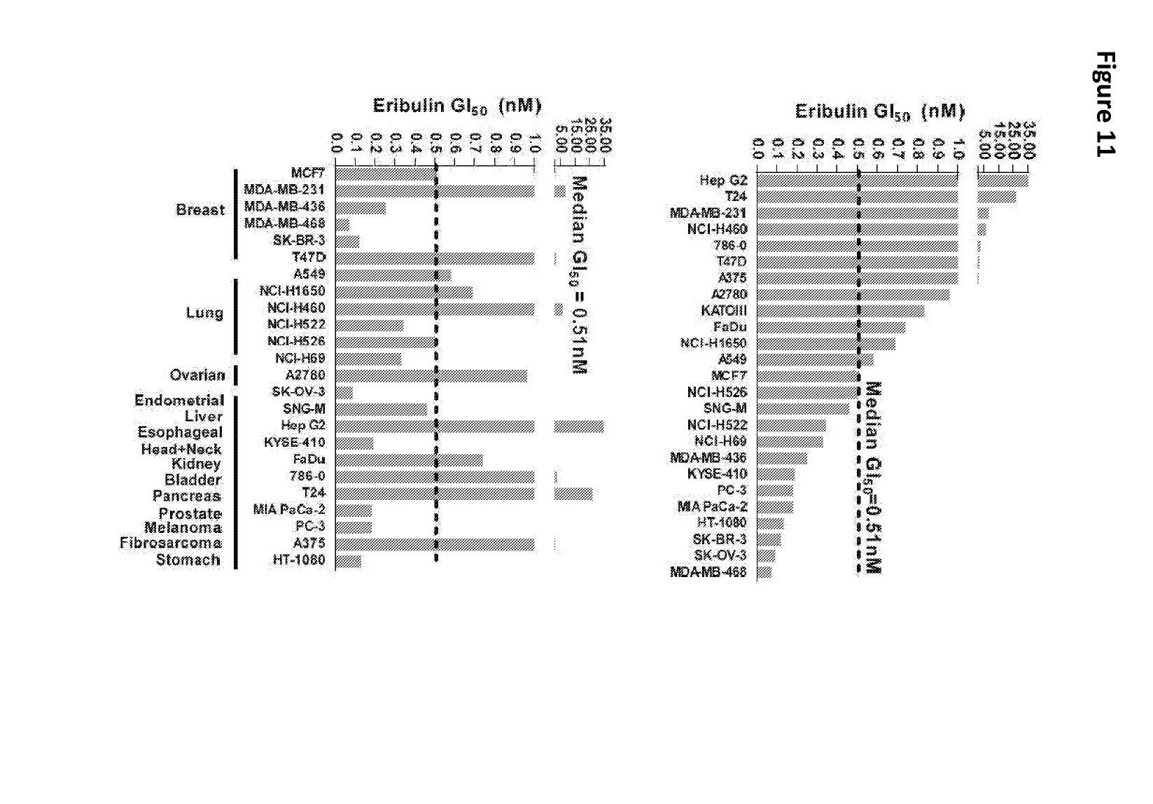

Eribulin had varying activity across a twenty-five cell line panel. Single agent activity was assessed using a three-fold, ten-point dose titration. Twenty-three cell lines were evaluated in 1536-well plate format. Two additional cell lines were evaluated in 384-well plate format. For cell lines where the GI₅₀ reached inhibition levels of greater than fifty percent, the median GI₅₀ was 0.51 nM (FIG. 11).

### Combination Screen Design

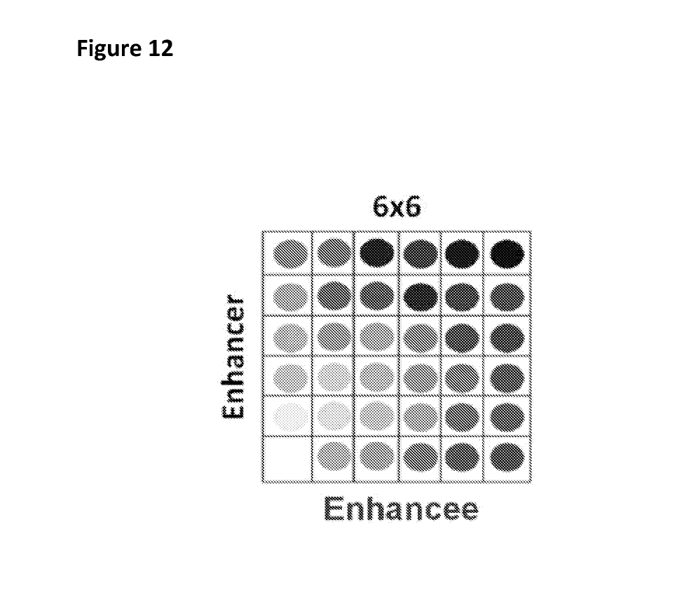

Combination analysis data was collected in a 6×6 dose matrix (FIG. 12). Twenty cell lines were screened in the 1536-well plate format, while five cell lines were screened in the 384-well plate format. Thirty-five enhancer compounds, including BKM-120, were combined with the enhancee molecule eribulin across the twenty-five cell line panel. In addition, twelve compounds were combined in selfcross analysis for each cell line. The starting concentration for the enhancee was centered at the EC90 for eribulin.

### Self-Cross-Based Combination Screen Analysis

In order to objectively establish hit criteria for the combination screen analysis, twelve compounds were selected to be self-crossed across the twenty-five cell line panel as a means to empirically determine a baseline additive, non-synergistic response. The identity of the twelve self-cross compounds was determined by selecting compounds with a variety of maximum response values and single agent dose response steepness. Those drug combinations which yielded effect levels that statistically superseded those baseline additivity values were considered synergistic.

The Synergy Score measure was used for the self-cross analysis. Synergy Scores of selfcrosses are expected to be additive by definition and, therefore, maintain a synergy score of zero. However, while some self-cross Synergy Scores are near zero, many are greater suggesting that experimental noise or non-optimal curve fitting of the single agent dose responses are contributing to the slight perturbations in the score. Overlay of the self-cross data for the twenty-five cell line panel demonstrates a global mean Synergy Score of 1.55. The global median across the cell line panel yielded a self-cross Synergy Score of 1.15, suggesting minimal influence by outlying values. Given the potential differences in cell line sensitivity to the eribulin combination activities, we chose to use a cell-line centric strategy for the self-cross based combination screen analysis, focusing on self-cross behavior in individual cell lines versus global review of the cell line panel activity. Combinations where the Synergy Score is greater than the mean self-cross plus two standard deviations (2σ's) or three standard deviations (3σ's) can be considered candidate synergies at the 95% and 99% confidence levels, respectively.

Synergy Scores that exceed the cell line specific mean self-cross threshold at 2σ's and 3σ's were determined. Loewe Volume cutoffs were also calculated on a per cell line basis at 2σ's and 3σ's above the mean. Based upon the statistical self-cross cutoffs, data was filtered for drug combinations displaying probable synergy across the cell line panel. The combination data was analyzed on a per cell line basis comparing the Loewe Volume and Synergy Scores to the respective self-cross cutoff's at 2σ's and 3σ's above the mean. Any Loewe Volume or Synergy Score greater than or equal to the self-cross cutoff was considered a hit.

### Everolimus

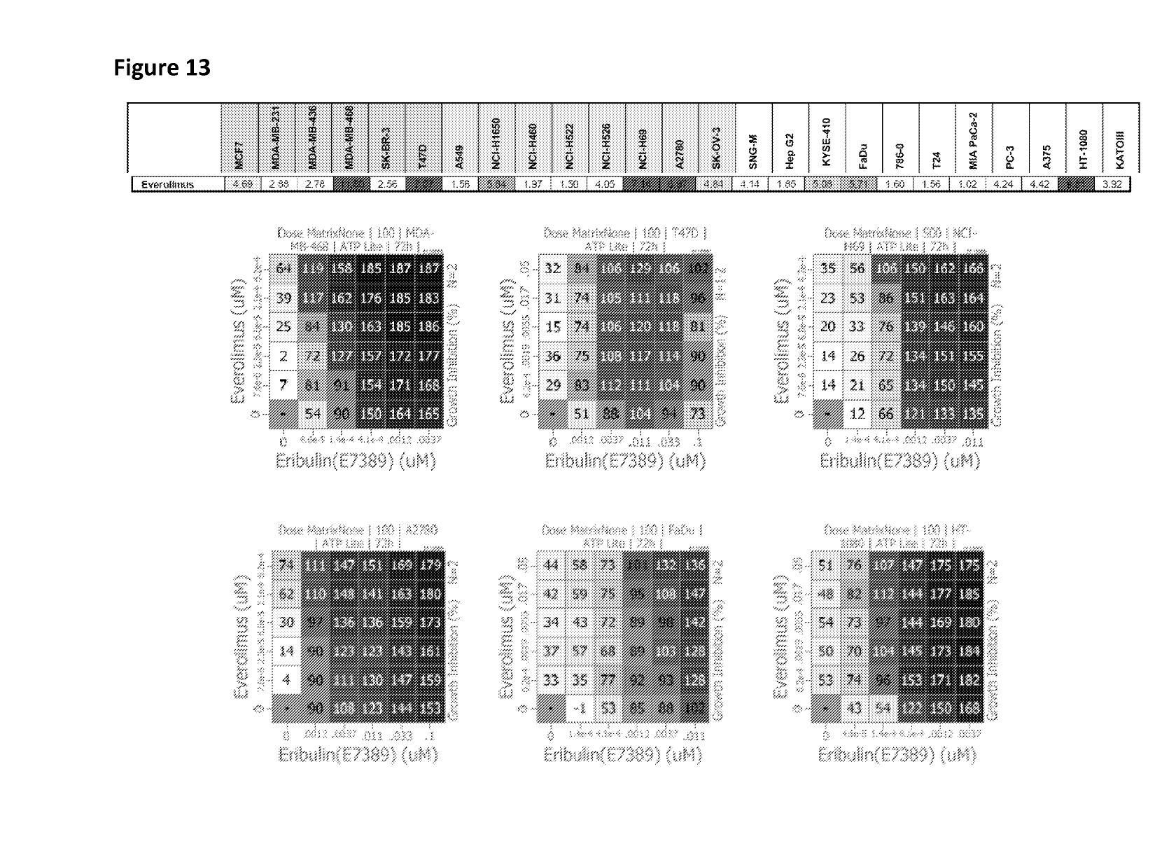

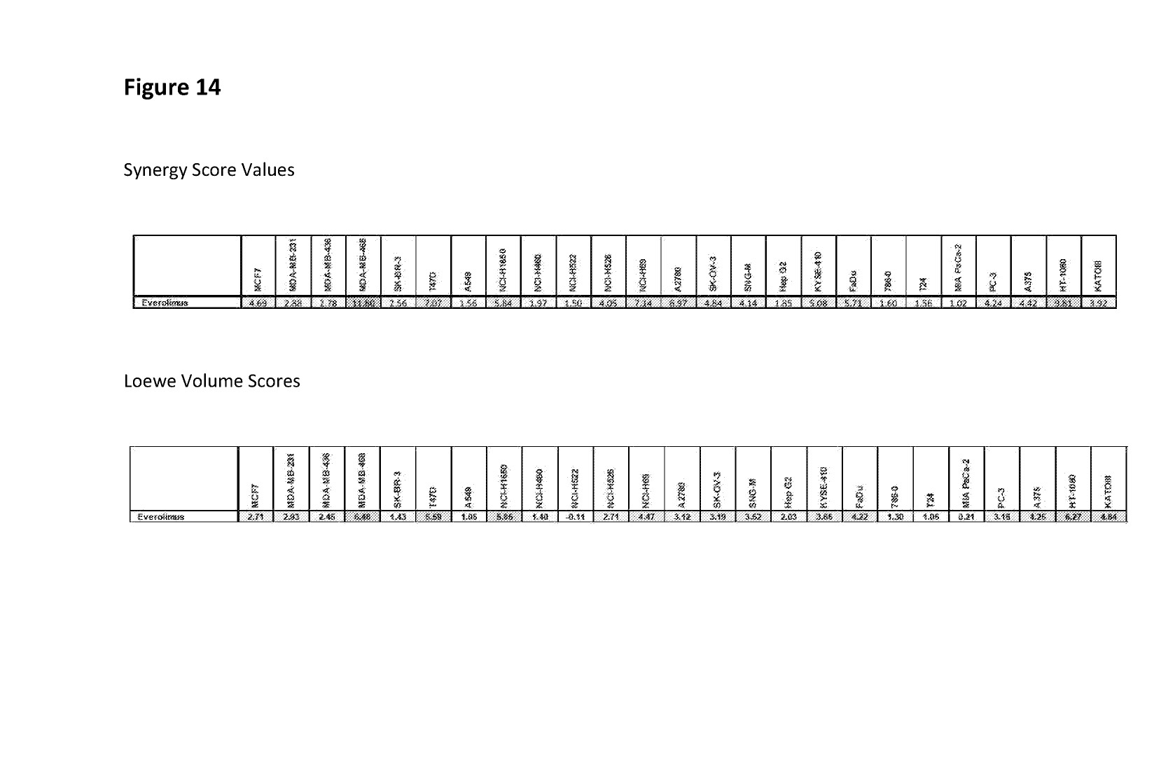

The phosphatidylinositol 3-kinase (PI3K) signaling pathway is a primary driver of cellular proliferation and a hallmark of many human cancers. Dysregulation of the PI3K pathway can be transformative by virtue of constitutive activation and, ultimately, stimulation of cellular proliferation and suppression of pro-apoptotic signaling. Everolimus is an allosteric mTOR (TORC1) inhibitor with an IC50 of 1.6-2.4 nM. As shown using a synergy score heat map (FIG. 13), good breadth of combination activity is observed across the cell line panel for everolimus. FIG. 14 provides synergy score values and Loewe Volume scores for everolimus.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features set forth herein.

All publications and patent applications mentioned in this specification are herein incorporated by reference to the same extent as if each independent publication or patent application was specifically and individually indicated as being incorporated by reference in their entirety.

Use of singular forms herein, such as “a” and “the,” does not exclude indication of the corresponding plural form, unless the context indicates to the contrary. Similarly, use of plural terms does not exclude indication of a corresponding singular form.

The invention is further described in the following numbered paragraphs.

1. A method for treating a subject having or at risk of developing cancer, the method comprising administering to the subject (i) eribulin or a pharmaceutically acceptable salt thereof, and (ii) an inhibitor of mammalian target of rapamycin (mTOR) or a pharmaceutically acceptable salt, hydrate, solvate, or amorphous solid thereof.

2. The method of paragraph 1, wherein said subject is a human patient.

3. The method of paragraph 1 or 2, wherein said subject is diagnosed with a cancer, in treatment for cancer, or in post-therapy recovery from cancer.

4. The method of any one of paragraphs 1 to 3, wherein said cancer is a primary tumor.

5. The method of any one of paragraphs 1 to 3, wherein said cancer is a metastasis.

6. The method of any one of paragraphs 1 to 5, wherein said cancer is a solid tumor.

7. The method of any one of paragraphs 1 to 6, wherein said cancer is selected from the group consisting of breast cancer, lung cancer, pancreatic cancer, primitive neuroectodermal tumors, lung cancer, ovarian cancer, endometrial cancer, pharyngeal cancer, esophageal cancer, and sarcoma.

8. The method of paragraph 7, wherein said cancer is selected from breast cancer and lung cancer.

9. The method of any one of paragraphs 1 to 8, wherein said pharmaceutically acceptable salt of eribulin is eribulin mesylate.

10. The method of any one of paragraphs 1 to 9, wherein said eribulin or said pharmaceutically acceptable salt thereof is administered by intravenous infusion.

11. The method of paragraph 10, wherein said intravenous infusion is for about 1 to about 20 minutes.

12. The method of paragraph 11, wherein said intravenous infusion is for about 2 to about 5 minutes.

13. The method of any one of paragraphs 1 to 12, wherein said eribulin or said pharmaceutically acceptable salt thereof is administered in an amount in the range of about 0.1 mg/m² to about 20 mg/m².

14. The method of paragraph 13, wherein said eribulin or said pharmaceutically acceptable salt thereof is administered in an amount of about 1.1 mg/m² or 1.4 mg/m².

15. The method of any one of paragraphs 1 to 14, wherein said eribulin or said pharmaceutically acceptable salt thereof is administered once on each of days 1 and 8 of a 21-day cycle, or each of days 1 and 15 of a 28-day cycle.

16. The method of any one of paragraphs 1 to 15, wherein said mTOR inhibitor is selected from the group consisting of everolimus, ridaforolimus, and temsirolimus, and pharmaceutically acceptable salts, hydrates, solvates, or amorphous solid thereof.

17. The method of any one of paragraphs 1 to 16, wherein said mTOR inhibitor is everolimus or a pharmaceutically acceptable salt, hydrate, solvate, or amorphous solid thereof.

18. The method of paragraph 17, wherein said everolimus or a pharmaceutically acceptable salt, hydrate, solvate, or amorphous solid thereof is administered orally.

19. The method of paragraph 18, wherein said everolimus or a pharmaceutically acceptable salt, hydrate, solvate, or amorphous solid thereof is administered in an amount in the range of about 0.1 mg to about 30 mg.

20. The method of paragraph 19, wherein said everolimus or a pharmaceutically acceptable salt, hydrate, solvate, or amorphous solid thereof is administered in an amount of about 10 mg.

21. The method of any one of paragraphs 1 to 20, wherein said mTOR inhibitor, or said pharmaceutically acceptable salt, hydrate, solvate, or amorphous solid thereof, is administered once daily during a 21-day cycle or a 28-day cycle.

22. The method of any one of paragraphs 1 to 21, wherein said eribulin or said pharmaceutically acceptable salt thereof, and said mTOR inhibitor, or said pharmaceutically acceptable salt, hydrate, solvate, or amorphous solid thereof, are administered substantially simultaneously or sequentially.

23. The method of paragraph 22, wherein said eribulin or said pharmaceutically acceptable salt thereof is administered prior to said mTOR inhibitor, or said pharmaceutically acceptable salt, hydrate, solvate, or amorphous solid thereof.

24. The method of any one of paragraphs 1 to 23, wherein said eribulin or said pharmaceutically acceptable salt thereof, and said mTOR inhibitor, or said pharmaceutically acceptable salt, hydrate, solvate, or amorphous solid thereof, are administered as sole anti-cancer agents.

25. The method of paragraph 24, wherein said pharmaceutically acceptable salt of eribulin is eribulin mesylate and/or said mTOR inhibitor is everolimus.

26. The method of any one of paragraphs 1 to 25, wherein said treating: (i) reduces the number of cancer cells; (ii) reduces tumor volume; (iii) increases tumor regression rate; (iv) reduces or slows cancer cell infiltration into peripheral organs; (v) reduces or slows tumor metastasis; (vi) reduces or inhibits tumor growth; (vii) prevents or delays occurrence and/or recurrence of the cancer and/or extends disease- or tumor-free survival time; (viii) increases overall survival time; (ix) reduces the frequency of treatment; and/or (x) relieves one or more of symptoms associated with the cancer.

27. A method for decreasing the size of a tumor in a subject, the method comprising administering to the subject (i) eribulin or a pharmaceutically acceptable salt thereof, and (ii) an mTOR inhibitor or a pharmaceutically acceptable salt, hydrate, solvate, or amorphous solid thereof.

28. The method of paragraph 27, wherein said mTOR inhibitor is everolimus or a pharmaceutically acceptable salt, hydrate, solvate, or amorphous solid thereof.

29. A kit for use in treating cancer or decreasing tumor size, the kit comprising (i) eribulin or a pharmaceutically acceptable salt thereof, and (ii) an mTOR inhibitor or a pharmaceutically acceptable salt, hydrate, solvate, or amorphous solid thereof.

30. The kit of paragraph 29, wherein said mTOR inhibitor is everolimus or a pharmaceutically acceptable salt, hydrate, solvate, or amorphous solid thereof.

31. The kit of paragraph 29 or 30, wherein said (i) eribulin or said pharmaceutically acceptable salt thereof, and said (ii) mTOR inhibitor or said pharmaceutically acceptable salt, hydrate, solvate, or amorphous solid thereof, are in dosage form.

What is claimed is:

1. A method for treating a subject having or at risk of developing cancer, the method comprising administering to the subject (i) eribulin or a pharmaceutically acceptable salt thereof, and (ii) an inhibitor of mammalian target of rapamycin (mTOR) or a pharmaceutically acceptable salt, hydrate, solvate, or amorphous solid thereof.

2. The method of claim 1, wherein said subject is a human patient.

3. The method of claim 1, wherein said subject is diagnosed with a cancer, in treatment for cancer, or in post-therapy recovery from cancer.

4. The method of claim 1, wherein said cancer is a primary tumor, a metastasis, or a solid tumor.

5. (canceled)

6. (canceled)

7. The method of claim 1, wherein said cancer is selected from the group consisting of breast cancer, lung cancer, pancreatic cancer, primitive neuroectodermal tumors, lung cancer, ovarian cancer, endometrial cancer, pharyngeal cancer, esophageal cancer, and sarcoma.

8. (canceled)

9. The method of claim 1, wherein said pharmaceutically acceptable salt of eribulin is eribulin mesylate.

10. The method of claim 1, wherein said eribulin or said pharmaceutically acceptable salt thereof is administered by intravenous infusion.

11. The method of claim 10, wherein said intravenous infusion is for about 1 to about 20 minutes, or is for about 2 to about 5 minutes.

12. (canceled)

13. The method of claim 1, wherein said eribulin or said pharmaceutically acceptable salt thereof is administered in an amount in the range of about 0.1 mg/m² to about 20 mg/m², or an amount of about 1.1 mg/m² or 1.4 mg/m².

14. (canceled)

15. The method of claim 1, wherein said eribulin or said pharmaceutically acceptable salt thereof is administered once on each of days 1 and 8 of a 21-day cycle, or each of days 1 and 15 of a 28-day cycle.

16. The method of claim 1, wherein said mTOR inhibitor is selected from the group consisting of everolimus, ridaforolimus, and temsirolimus, and pharmaceutically acceptable salts, hydrates, solvates, or amorphous solid thereof.

17. (canceled)

18. The method of claim 16, wherein said mTOR inhibitor is everolimus or a pharmaceutically acceptable salt, hydrate, solvate, or amorphous solid thereof, and is administered orally.

19. The method of claim 18, wherein said everolimus or a pharmaceutically acceptable salt, hydrate, solvate, or amorphous solid thereof is administered in an amount in the range of about 0.1 mg to about 30 mg, or an amount of about 10 mg.

20. (canceled)

21. The method of claim 1, wherein said mTOR inhibitor, or said pharmaceutically acceptable salt, hydrate, solvate, or amorphous solid thereof, is administered once daily during a 21-day cycle or a 28-day cycle.

22. The method of claim 1, wherein said eribulin or said pharmaceutically acceptable salt thereof, and said mTOR inhibitor, or said pharmaceutically acceptable salt, hydrate, solvate, or amorphous solid thereof, are administered substantially simultaneously or sequentially.

23. The method of claim 22, wherein said eribulin or said pharmaceutically acceptable salt thereof is administered prior to said mTOR inhibitor, or said pharmaceutically acceptable salt, hydrate, solvate, or amorphous solid thereof.

24. The method of claim 1, wherein said eribulin or said pharmaceutically acceptable salt thereof, and said mTOR inhibitor, or said pharmaceutically acceptable salt, hydrate, solvate, or amorphous solid thereof, are administered as sole anti-cancer agents, and optionally wherein said pharmaceutically acceptable salt of eribulin is eribulin mesylate and/or said mTOR inhibitor is everolimus or or a pharmaceutically acceptable salt, hydrate, solvate, or amorphous solid thereof.

25. (canceled)

26. The method of claim 1, wherein said treating: (i) reduces the number of cancer cells; (ii) reduces tumor volume; (iii) increases tumor regression rate; (iv) reduces or slows cancer cell infiltration into peripheral organs; (v) reduces or slows tumor metastasis; (vi) reduces or inhibits tumor growth; (vii) prevents or delays occurrence and/or recurrence of the cancer and/or extends disease- or tumor-free survival time; (viii) increases overall survival time; (ix) reduces the frequency of treatment; and/or (x) relieves one or more of symptoms associated with the cancer.

27. A method for decreasing the size of a tumor in a subject, the method comprising administering to the subject (i) eribulin or a pharmaceutically acceptable salt thereof, and (ii) an mTOR inhibitor or a pharmaceutically acceptable salt, hydrate, solvate, or amorphous solid thereof, optionally wherein said mTOR inhibitor is everolimus or a pharmaceutically acceptable salt, hydrate, solvate, or amorphous solid thereof.

28. (canceled)

29. A kit for use in treating cancer or decreasing tumor size, the kit comprising (i) eribulin or a pharmaceutically acceptable salt thereof, and (ii) an mTOR inhibitor or a pharmaceutically acceptable salt, hydrate, solvate, or amorphous solid thereof, optionally wherein said mTOR inhibitor is everolimus or a pharmaceutically acceptable salt, hydrate, solvate, or amorphous solid thereof, and further optionally wherein said (i) eribulin or said pharmaceutically acceptable salt thereof, and said (ii) mTOR inhibitor or said pharmaceutically acceptable salt, hydrate, solvate, or amorphous solid thereof, are in dosage form.

30. (canceled)

31. (canceled)
